(19) United States
(12) Patent Application Publication
(10) Pub. No.: US 2023/0373962 A1
(43) Pub. Date: Nov. 23, 2023

(54) SUPRAMOLECULAR MOLECULES FOR THE TREATMENT OF CANCER

(71) Applicant: The University of Birmingham, Birmingham (GB)

(72) Inventor: Michael John Hannon, Birmingham (GB)

(21) Appl. No.: 18/028,238

(22) PCT Filed: Sep. 28, 2021
(86) PCT No.: PCT/EP2021/076679

§ 371 Date: Mar. 24, 2023

(30) Foreign Application Priority Data
Sep. 28, 2020 (GB) 2015310.2
Nov. 13, 2020 (GB) 2017911.5

(51) Int. Cl. C07D 401/14; C08B 37/16; C08G 83/00

(52) U.S. Cl. CPC C07D 401/14; C08B 37/0015; C08G 83/007

(57) ABSTRACT

The present invention provides rotaxanes comprising a macrocycle surrounding an ion comprising a blocking group capable of trapping the macrocycle. Also provided are methods of making such rotaxanes and a method of removing the blocking group from the rotaxane, thereby allowing the ion to escape the macrocycle. The ions have been found to have anticancer, antibacterial and/or antiviral properties. Accordingly, the rotaxanes of the invention may be useful in the treatment of cancer, bacterial and/or viral diseases.

### FIELD OF THE INVENTION

The present invention provides rotaxanes comprising a macrocycle surrounding an ion of formula [M_{z}L_{z1}]^{zn+}, wherein said ion comprises a blocking group capable of trapping the macrocycle. Also provided herein are methods of making such rotaxanes and a method of removing the blocking group from the rotaxane, thereby allowing the ion to escape the macrocycle. The ions have been found to have anticancer, antibacterial and/or antiviral properties. Accordingly, the rotaxanes of the invention may be useful in the treatment of cancer, bacterial and/or viral diseases.

### BACKGROUND OF THE INVENTION

Rotaxanes are mechanically interlocked molecular structures comprising a molecular strand threaded through a macrocycle and locked in place by blocking groups on the ends of the strand. The blocking groups are larger than the internal diameter of the macrocycle and thus prevent dissociation (unthreading) of the molecular strand from the macrocycle.

Rotaxanes have been formed by threading molecular strands through a variety of different classes of macrocycle (see Stoddart, J. F, Angew. Chem. Int. Ed., 2017, 56, 11094-125; Sauvage, J.-P, Angew. Chem. Int. Ed., 2017, 56, 11080-93; and Zhang, L., Marcos, V., Leigh, D. A., Proc. Natl. Acad. Sci., USA, 2018, 115, 9397-9404). Applications of rotaxanes range from protecting peptides from rapid enzymatic degradation, to creating materials with switchable surface properties (see Pairault, N. et al., C. R. Chimie, 2016, 19, 103e112). However, the threads used, to date, have been principally linear, 1-dimensional or 2-dimensional, organic, covalent molecules.

Self-assembled metallo-supramolecular helicates offer quite large nanoscale surfaces of defined topography, of comparable scale to key structures found in biomolecules. These external surfaces have been used to access unique DNA- and RNA-recognition properties, demonstrating with X-ray crystal structures the binding of cylindrical metallo-helicates in the heart of DNA and RNA 3-way junction structures, where the nucleic acid bases pi-stack perfectly onto the external aromatic surfaces of the metallo-helicates (see Boer, D. R et al., Angew. Chem. Intl. Ed., 2010, 49, 2336-2339 and Cerasino, L., Hannon, M. J., Sletten, E., Inorg. Chem., 2007, 46, 6245-6251). Nucleic acid junction structures are targets found both in biology and nucleic acid nanoscience. Junction-binding self-assembled metallo-supramolecular helicates arrest the proliferation of cancer cells, bacteria and viruses (see Hotze, A. C. G. et al., Chem. Biol., 2008, 15, 1258-1267; A. D. Richards et al., Int. J. Antimicrob. Agents, 2009, 33, 469-472; and Cardo, L. et al., Sci. Rep. 2018, 8, 13342).

The ability to deliver a non-active self-assembled metallo-supramolecular helicate and turn it into an active self-assembled metallo-supramolecular helicate at a target site could be effective in controlling the specificity of the self-assembled metallo-supramolecular helicate in the body. Thus, the present invention aims to create new types of rotaxanes and pseudo-rotaxanes in which the thread is not a simple molecular chain but a three dimensional, self-assembled, cylindrical metallo-supramolecular object. Pseudo-rotaxanes have been created by clipping macrocycles around carbon nanotubes (see Perez, E. M., Chem. Eur. J., 2017, 23, 12681-12689 and Barrejón, M., Mateo-Alonso, A., Prato, M., Eur. J. Org. Chem., 2019, 3371-3383) and a rotaxane with a spherical fullerene in the centre has recently been reported (see Xu, Y. et al., J. Am. Chem. Soc., 2018, 140, 13413-13420). However, an interlocked rotaxane assembled around a three-dimensional cylindrical metallo-supramolecular object rather than a linear thread has not, to the best of our knowledge, been reported, nor has rotaxanation in DNA-binding agents.

### SUMMARY OF THE INVENTION

The inventors have found that an ion of formula [M_{z}L_{z1}]^{zn+} may be surrounded with a macrocycle, which macrocycle may be trapped around the ion by blocking groups bound to the ion, resulting in the formation of a rotaxane. Forming such rotaxanes is a surprisingly effective way of switching the DNA-binding properties of the ions comprised within the rotaxane. In other words, the inventors have found that trapping the ions disclosed herein within a rotaxane switches off the biological activity of the ion. The biological activity may then be switched back on by removing the blocking group from the rotaxane for example, by the methods disclosed herein, allowing the ion to de-thread (diffuse away from the macrocycle).

Viewed from a first aspect, there is provided a rotaxane comprising a macrocycle and an ion of formula [M_{z}L_{z1]}^{zn+}, wherein M is a metal ion, or combination of ions of oxidation state n⁺, z is 2 to 4, z1 is 2 to 6 and L is a ligand of formula (I):

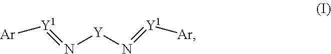

wherein Y¹ is independently CR¹ or N;
R¹ is independently selected from H, C₁-C₄alkyl, amino, phenyl or C₃-C₅heteroaryl, wherein the phenyl or C₃-C₅heteroaryl is optionally substituted with any one or a combination selected from the group consisting of C₁-C₄alkoxy, C₁-C₄alkyleneC₁-C₄alkanoate, C₁-C₄alkanoate, hydroxy, C₁-C₄alkylol, carboxy, C₁-C₄alkyl, halo, cyano, nitrite, C₁-C₄haloalkyl, C₁-C₄alkylthio, C₁-C₄alkyleneC₁-C₄alkylthio, C₁-C₄alkylsulfonate, diC₁-C₄alkylamino and C₁-C₄alkynyl;
each Ar is positioned at either end of the ion of formula [M_{z}L_{z1}]^{zn+} and is independently a C₃-C₉heteroaryl comprising at least one nitrogen atom, is optionally substituted with one or more blocking groups capable of trapping the macrocycle, with the proviso that each end of the ion comprises at least one blocking group, and is optionally substituted with one or more substituents selected from the group consisting of hydroxy, C₁-C₄alkylol, carboxy, C₁-C₄alkanoate, C₁-C₄alkyl, C₁-C₄alkoxy, halo, cyano, nitrite, C₁-C₄haloalkyl, C₁-C₄alkylthio, C₁-C₄alkyleneC₁-C₄alkylthio, C₁-C₄alkylsulfonate, diC₁-C₄alkylamino and C₁-C₄alkynyl;
N—Y—N is N—N or is any one selected from the group consisting of (Ia) to (Ic):

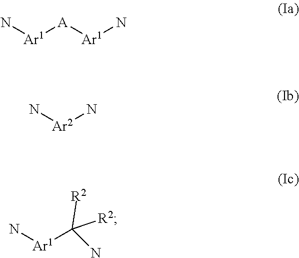

wherein A is NH, S, SO₂, O, (CH₂)₁₋₄, CHR₂, CR²₂ or NR²;
Ar¹ is phenyl optionally substituted one or more times with R³;
Ar² is phenyl or biphenyl optionally substituted one or more times with R³; and
R² and R³ are independently any one or a combination selected from the group consisting of H, hydroxy, C₁-C₄alkylol, carboxy, C₁-C₄alkanoate, C₁-C₄alkyl, halo, cyano, nitrite, C₁-C₄haloalkyl, C₁-C₄alkylthio, C₁-C₄alkyleneC₁-C₄alkylthio, C₁-C₄alkylsulfonate, diC₁-C₄alkylamino and C₁-C₄alkynyl.

Viewed from a second aspect, there is provided a method of synthesising a rotaxane, the method comprising:
(i) contacting a macrocycle and an ion of formula [M_{z}L_{z1}]^{zn+} to produce a pseudo-rotaxane, wherein the ion of formula [M_{z}L_{z1}]^{zn+} is as defined in the first aspect, with the proviso that each Ar is not substituted with the one or more blocking groups capable of trapping the macrocycle; and
(ii) reacting the pseudo-rotaxane with a compound of formula b-X to form the rotaxane, wherein b is a blocking group capable of trapping the macrocycle and X is a leaving group, with the proviso that each end of the ion of formula [M_{z}L_{z1}]^{zn+} of the rotaxane comprises at least one blocking group.

Viewed from a third aspect, there is provided a method of synthesising a rotaxane, the method comprising contacting a macrocycle and an ion of formula [M_{z}L_{z1}]^{zn+} at temperatures of about 50 to about 100° C., wherein the ion of formula [M_{z}L_{z1}]^{zn+} is as defined in the first aspect.

As described above, the inventors have found that the biological activity of the ion of formula [M_{z}L_{z1}]^{zn+} is switched off when the ion is comprised within a rotaxane. The biological activity may then be switched back on by removing blocking group(s) from the rotaxane, allowing the ion to diffuse away from the macrocycle.

Accordingly, viewed from a fourth aspect, there is provided a method of removing blocking group(s) from the rotaxane of the first aspect, the method comprising reacting the rotaxane with hydrogen, photo-irradiating the rotaxane, contacting the rotaxane with an enzyme, reacting the rotaxane with a reducing agent or reacting the rotaxane with water or thiols.

Viewed from a fifth aspect, there is provided an ion of formula [M_{z}L_{z1}]^{zn+}, as defined in the first aspect, wherein the blocking groups are capable of reversibly trapping a macrocycle.

Viewed from a sixth aspect, there is provided a pharmaceutical formulation comprising the rotaxane of the first aspect and one or more pharmaceutically acceptable excipients.

The ions comprised within the rotaxane have been found to have anticancer, antibacterial and/or antiviral properties. Accordingly, viewed from a seventh aspect, there is provided a rotaxane of the first aspect or a pharmaceutical formulation of the sixth aspect for use as a medicament, such as for use in the treatment of any one or a combination selected from the group consisting of cancer, a viral disease and a bacterial disease.

Viewed from an eighth aspect, there is provided a method of treatment, such as a method of treating cancer, a viral disease and/or a bacterial disease, comprising administering a therapeutically effective amount of a rotaxane of the first aspect or a pharmaceutical formulation of the sixth aspect to a subject.

### BRIEF DESCRIPTION OF THE FIGURES

FIG. 1 is a schematic diagram of a conventional (linear thread) rotaxane (left) and the cylinder rotaxane developed herein (right).

FIG. 2 depicts the ligands exemplified together with the structure of the [Ni₂L₃]⁴⁺ cylinder (CSD-NITBIB) and cucurbit[10]uril (from CSD-LAZPIM and illustrating the flexibility and potential ellipsoidal distortions CB[10] can accommodate in response to guests).

FIG. 3 is a 500 MHz ¹H NMR spectra of the [Fe₂L₃]⁴⁺ cylinder (0.5 mM) (a) alone and (b) with 1 equivalent of CB[10] in D₂O illustrating the shifts caused by binding. (c) DOSY NMR spectra (stacked) showing the change in diffusion coefficient on binding consistent with formation of a single species of increased hydrodynamic radius. (d) DFT model of the cylinder+CB[10] also showing the experimentally observed NOEs from cylinder H-im & H-3 to a CH₂ proton on the rim of the CB[10].

FIG. 4 depicts proton NMR spectra of the [Fe₂L₃]⁴⁺ cylinder (top) alone and (bottom) with 0.9 equivalents of CB[10] in D₂O illustrating the shifts of the bound and unbound cylinder. The peaks b/d and a/c are in exchange through phenyl ring spinning; the apparent sharpening on CB[10] binding could be due to a change in ring spinning rate when inside the cavity or arise from a change in their chemical shift separations of the two exchanging proton environments.

FIG. 5 depicts two pairs of 400 MHz Proton NMR spectrum in D₂O of the [Ni₂L₃]⁴⁺ cylinder with 1 equivalent of CB[10] (upper) and alone (lower). The bottom pair is an expansion of the 0-10 ppm region, highlighting the CB[10] peaks.

FIG. 6 is (a) PAGE gel showing that the [Ni₂L₃CB10]⁴⁺ complex stabilises DNA 3WJ in the same way as [M₂L₃]⁴⁺ cylinders alone. (b) CD and (c) LD spectra of ct-DNA (100 μM DNA concentration; 10 mM sodium chloride; 1 mM sodium cacodylate) with [Ni₂L₃·CB10]⁴⁺. R values represent DNA base pairs/complex. The spectra show binding and DNA coiling and are analogous to those obtained with free [Ni₂L₃]⁴⁺ (d) MTT cell survival assay (72 hours treatment of A2780 cells), demonstrating that [Ni₂L₃·CB10]⁴⁺ and [Ni₂L₃]⁴⁺ have similar effects on cell survival. (e) Cell uptake studies demonstrating that CB[10] does not affect the cell uptake.

FIG. 7 depicts the synthetic strategy disclosed herein to create a rotaxanated cylinder.

FIG. 8 is (a) mass Spectrum of the [Ni₂L″₃CB10]⁴⁺ rotaxane (b) ¹H NMR Spectrum (0-10 ppm shown only) of the [Ni₂L″₃·CB10]⁴⁺ rotaxane and comparison with imidazole pseudo-rotaxane [Ni₂L′₃CB10]⁴⁺ showing presence of CB[10] and the alkylpyridine groups on the modified cylinder.

FIG. 9 depicts combined and superimposed views of Molecular Dynamics simulations of the pseudo-rotaxanated [Ni₂L′₃·CB10]⁴⁺ (left) and proper rotaxanated [Ni₂L″₃·CB10]⁴⁺ (right) complexes showing the free rotation of the [Ni₂L′₃]⁴⁺ cation in the CB[10] and the more restricted motion caused by the picolyl groups for [Ni₂L″₃]⁴⁺; in the 1 μs timescale the rotaxanated cylinder does not rotate. Hydrogen atoms are omitted for clarity.

FIG. 10 is (a) PAGE gel showing that the [Ni₂L′₃]⁴⁺ and [Ni₂L″₃]⁴⁺ complexes stabilise DNA 3WJ but the rotaxanated [Ni₂L″₃·CB10]⁴⁺ cylinder does not. (b) CD and (c) LD spectra of ct-DNA (100 μM DNA concentration; 10 mM sodium chloride; 1 mM sodium cacodylate) with [Ni₂L″₃]⁴⁺ and (d) the corresponding CD and (e) LD spectra of ct-DNA with [Ni₂L″₃·CB10]⁴⁺. R values represent DNA base pairs/complex.

FIG. 11 comprises electrospray mass spectra of a competition experiment treating a partially alkylated rotaxanate mix [Ni₂L″₃·CB10]⁴⁺ with one equivalent of [Fe₂L₃]⁴⁺ in aqueous methanol (1:1) showing the dethreading of partially alkylated cylinders and the formation of pseudo-rotaxane [Fe₂L₃·CB10]⁴⁺. No peaks corresponding to dethreading of fully alkylated (hexa-alkylated) cylinders were observed.

FIG. 12 is a competition experiment between a partially alkylated rotaxane mix and [Fe₂L₃]⁴⁺ studied by electrospray mass spectrometry. Additional time points beyond those shown in FIG. 11. Note that no hexa-alkylated cylinder ions are observed in the experiment. Rot3 and Rot 4 decrease (˜30 and 20% over 28 h) compared to the other Rot tetracations

FIG. 13 shows (a) Competition experiment between a partially alkylated rotaxane mix and memantine studied by electrospray mass spectrometry. (b) Gel electrophoresis study of the partially alkylated rotaxane mix with the 3WJ DNA strands showing that—consistent with the mass spectrometry competition results—cylinders with low alkylation numbers can dethread from the rotaxane (kinetic rotaxanes) and bind and stabilise the 3WJ.

FIG. 14 is a Mass spectrum (electrospray) of the reaction of pre-alkylated [Ni₂(L″)₃]Cl₄ with CB[10] at 373K confirming direct formation of the rotaxane, either through threading at high temperature or (more likely) cylinder dissociation and re-formation. Small amounts of non-rotaxanated cylinder are also observed.

FIG. 15 shows the structure of exemplar ligands used in rotaxanated complexes and referred to in the text

FIG. 16 is Mass spectra of DNB rotaxane [Ni₂(L^{dnb})₃·CB10]⁴⁺ with 10 mM mercaptoethanol (as Thiol) in methanol demonstrating cleavage of the stoppers and release of the cylinder (Cyl) from the rotaxane species (R) over time (t). Subscripts on Cyl and R peak labels indicate number of attached stoppers (0-6) while superscripts show charge on the ion (4+, 2+).

FIG. 17 shows PAGE Gel electrophoresis of DNA 3-way junction (3WJ) binding following rotaxane decapping in water/buffer with 10mM of decaping agent. The gel shows that the rotaxanated [Ni₂(L^{dnb})₃·CB10]⁴⁺ cylinder does not stabilise DNA 3WJ but once decapped, the cylinders can be released and their 3WJ binding is enabled. Upper band is 3WJ and the lower band the individual single strands.

### DETAILED DESCRIPTION OF THE INVENTION

Throughout this specification, one or more aspects of the invention may be combined with one or more features described in the specification to define distinct embodiments of the invention.

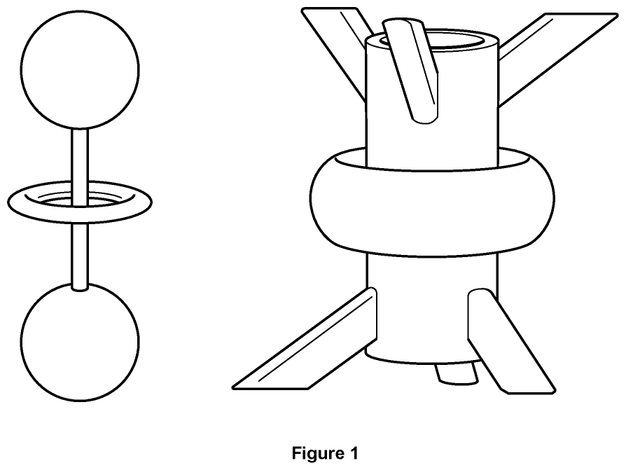

As described above, the inventors have found that forming the rotaxanes of the invention is a surprisingly effective way of switching the DNA-binding properties of the ions comprised within the rotaxane. The inventors have taken advantage of the three-dimensional cylindrical surface of the ions of formula [M_{z}L_{z1}]^{zn+} and have replaced the classic dumb-bell design of the molecular threads of known rotaxanes with ‘roots or branches’ emanating from the three-dimensional ‘trunk’ of the axle (the ion of formula [M_{z}L_{z1}]^{zn+}), in order to mechanically constrain the macrocycle of the rotaxane (see FIG. 1).

In the discussion that follows, reference is made to a number of terms, which are to be understood to have the meanings provided below, unless a context indicates to the contrary. The nomenclature used herein for defining compounds, in particular the compounds described herein, is intended to be in accordance with the rules of the International Union of Pure and Applied Chemistry (IUPAC) for chemical compounds, specifically the “IUPAC Compendium of Chemical Terminology (Gold Book)” (see A. D. Jenkins et al., Pure & Appl. Chem., 68, 2287-2311 (1996)). For the avoidance of doubt, if an IUPAC rule is contrary to a definition provided herein, the definition herein is to prevail.

The term “comprising” or variants thereof will be understood to imply the inclusion of a stated element, integer or step, or group of elements, integers or steps, but not the exclusion of any other element, integer or step, or group of elements, integers or steps.

The term “consisting” or variants thereof will be understood to imply the inclusion of a stated element, integer or step, or group of elements, integers or steps, and the exclusion of any other element, integer or step or group of elements, integers or steps.

The term “about” herein, when qualifying a number or value, is used to refer to values that lie within ±5% of the value specified. For example, if a temperature is defined as about 50° C. to about 100° C., temperatures of 47.5° C. to 105° C. are included.

Oxidation state of an ion is defined herein as the charge of the ion, after ionic approximation of its heteronuclear bonds.

The term “alkyl” is well known in the art and defines univalent groups derived from alkanes by removal of a hydrogen atom from any carbon atom, wherein the term “alkane” is intended to define acyclic branched or unbranched hydrocarbons having the general formula CₙH₂ₙ₊₂, wherein n is an integer ≥1. C₁-C₄alkyl refers to any one selected from the group consisting of methyl, ethyl, n-propyl, iso-propyl, n-butyl, sec-butyl, iso-butyl and tert-butyl.

An amino may be a primary (—NH₂), secondary (—NRH) or tertiary (—NR₂) amino, where R is, or each R is independently, a hydrocarbyl group. Often, where the amino is a secondary or tertiary amino, it is a C₁-C₄alkylamino or diC₁-C₄alkylamino.

The term “hydrocarbyl” defines univalent groups derived from hydrocarbons by removal of a hydrogen atom from any carbon atom, wherein the term “hydrocarbon” refers to compounds consisting of hydrogen and carbon only.

The term “heteroaryl” defines a group derived from a heteroarene by removal of a hydrogen atom from a ring carbon or heteroatom, wherein a heteroarene is a monocyclic or polycyclic aromatic hydrocarbon comprising one or more heteroatoms.

The term “alkoxy” defines univalent groups derived from alcohols by removal of the hydrogen atom from the oxygen atom of the alcohol, wherein the term “alcohol” is intended to define compounds derived from alkanes, wherein a hydrogen atom bonded to any carbon atom is substituted for a hydroxy group, —OH. C₁-C₄alkoxy refers to any one selected from the group consisting of methoxy, ethoxy, n-propoxy, iso-propoxy, n-butoxy, sec-butoxy, iso-butoxy and tert-butoxy.

The term “alkylenealkanoate” defines univalent groups of formula —CₙH₂ₙOC(O)CₙH₂ₙ₊₁, e.g. ethylene propanoate, derived from esters by removal of a hydrogen atom from the alkyl group of the ester, wherein the term “ester” is intended to define compounds derived from an oxoacid and an alcohol. Where the alkylenealkanoate is a C₁-C₄alkyleneC₁-C₄alkanoate, the C₁-C₄alkylene is any one selected from the group consisting of methylene, ethylene, n-propylene, iso-propylene, n-butylene, sec-butylene, iso-butylene and tert-butylene, and the C1-C₄alkanoate is any one selected from the group consisting of methanoate, ethanoate, n-propanoate, iso-propanoate, n-butanoate, sec-butanoate, iso-butanoate and tert-butanoate.

The term “alkanoate” defines univalent groups of formula —OC(O)CₙH₂ₙ₊₁, e.g. propanoate, derived from carboxylic acids by removal of a hydrogen atom from the hydroxy group of the carboxylic acid.

The term “alkylol” defines univalent groups of formula —CₙH₂ₙOH, e.g. propylol, derived from alcohols by removal of a hydrogen atom from the alkyl group of the alcohol.

The term “haloalkyl” defines univalent groups derived from alkyls by substitution of one or more hydrogen atoms from the alkyl with halo atoms. Halo includes fluoro, chloro, bromo, iodo and astato. In some embodiments, halo includes fluoro, chloro, bromo and iodo. In some embodiments, the haloalkyl is a fluoroalkyl.

The term “alkylthio” defines univalent groups derived from thiols by removal of the hydrogen atom from the sulfur atom of the thiol, wherein the term “thiol” is intended to define compounds derived from alkanes, wherein a hydrogen atom bonded to any carbon atom is substituted for a sulfanyl group, —SH. In other words, alkylthio is the sulfur analogue of alkoxy. C₁-C₄alkylthio refers to any one selected from the group consisting of methylthio, ethylthio, n-propylthio, iso-propylthio, n-butylthio, sec-butylthio, iso-butylthio and tert-butylthio.

The term “alkylenealkylthio” defines univalent groups of formula —CₙH₂ₙSCₙH₂ₙ₊₁, e.g. methyl ethylene sulphide, derived from dialkylsulfides, wherein any hydrogen atom is removed from one of the alkyl groups.

The term “alkylsulfonate” defines univalent groups of formula —OSO₂CₙH₂ₙ₊₁, e.g. methylsulfonate, derived from alkylsulfonic acids by removal of a hydrogen atom from the hydroxy group of the alkylsulfonic acid. C₁-C₄alkylsulfonate refers to any one selected from the group consisting of methylsulfonate, ethylsulfonate, n-propylsulfonate, iso-propylsulfonate, n-butylsulfonate, sec-butylsulfonate, iso-butylsulfonate and tert-butylsulfonate.

The term “alkynyl” defines univalent groups derived from alkynes by removal of a hydrogen atom from any carbon atom, wherein the term “alkyne” is intended to define acyclic branched or unbranched hydrocarbons having one carbon-carbon triple bond and the general formula CₙH₂ₙ₋₂, where n is an integer ≥2. C₂-C₄alkynyl refers to any one selected from the group consisting of ethynyl, prop-1-ynyl, prop-2-ynyl, but-1-ynyl, but-2-ynyl, but-3-ynyl, and 1-methyl-prop-2-ynyl.

The term “heteroarylalkylene” defines univalent groups derived from a heteroarene by substitution of a hydrogen atom from a ring carbon or heteroatom with an alkylene. The term “alkylene” defines divalent groups derived from an alkane by removal of any two hydrogen atoms from the alkane. Examples of heteroarylalkylene include 4-methylenepyridine (known as picolyl) and 3-methylenequinoline.

The term “phenylalkylene” defines univalent groups derived from benzene by substitution of a hydrogen atom from a ring carbon with an alkylene. Examples of phenylalkylene include benzyl and ethylenebenzene.

The term “treatment” defines the therapeutic treatment of a human or non-human animal, in order to impede or reduce or halt the rate of the progress of the condition, or to ameliorate or cure the condition. Prophylaxis of the condition as a result of treatment is also included. References to prophylaxis are intended herein not to require complete prevention of a condition: its development may instead be hindered through treatment in accordance with the invention. Typically, treatment is not prophylactic, and the rotaxane or composition comprising the rotaxane is administered to a patient having a diagnosed or suspected condition.

By an “effective amount” herein defines an amount of the compound or composition of the invention that is sufficient to impede the noted diseases and thus produces the desired therapeutic or inhibitory effect.

The term “stereoisomer” is used herein to refer to isomers that possess identical molecular formulae and sequence of bonded atoms, but which differ in the arrangement of their atoms in space.

The term “enantiomer” defines one of a pair of molecular entities that are mirror images of each other and non-superimposable, i.e. cannot be brought into coincidence by translation and rigid rotation transformations. Enantiomers are chiral molecules, i.e. are distinguishable from their mirror image.

The term “racemic” is used herein to pertain to a racemate. A racemate defines a substantially equimolar mixture of a pair of enantiomers.

The term “diastereoisomers” (also known as diastereomers) defines stereoisomers that are not related as mirror images.

The term “solvate” is used herein to refer to a complex comprising a solute, such as a rotaxane or a composition comprising a rotaxane disclosed herein and a solvent. If the solvent is water, the solvate may be termed a hydrate, for example a mono-hydrate, di-hydrate, tri-hydrate etc, depending on the number of water molecules present per molecule of substrate.

The term “isotope” is used herein to define a variant of a particular chemical element, in which the nucleus necessarily has the same atomic number but has a different mass number owing to it possessing a different number of neutrons.

The term “pharmaceutically acceptable excipient” defines substances other than a rotaxane, which are included in a pharmaceutical product.

As described above, rotaxanes are mechanically interlocked molecular structures comprising a molecular strand threaded through a macrocycle and locked in place by blocking groups on the ends of the strand. The rotaxanes disclosed herein differ from classic rotaxanes in that the molecular thread is an ion of formula [M_{z}L_{z1}]^{zn+}, i.e. a three-dimensional cylinder rather than a linear thread.

A macrocycle is defined herein to be a cyclic macromolecule comprising a twelve or more membered ring. The macrocycles disclosed herein comprise a cavity in which an ion of formula [M_{z}L_{z1}]^{zn+} may reside.

The rotaxanes disclosed herein comprise blocking groups (at least one blocking group at each end of the ion of formula [M_{z}L_{z1}]^{zn+}) capable of trapping the macrocycle (by which is meant that the movement of the macrocycle past the blocking group(s) is hindered, and the macrocycle is thus kinetically locked in place). For the avoidance of doubt, the macrocycle need not be trapped by the blocking groups indefinitely. Rather, the macrocycle may be trapped (in aqueous solution at 20° C., pH 7, and at ambient pressure (e.g. 1 bar)) by the blocking groups for at least two hours, at least a day, at least one week, at least two weeks, at least one month, at least three months, at least six months or at least one year. These time frames refer to the half-life of the rotaxane, i.e. the time it takes for half of the rotaxanes to disassociate, i.e. for the macrocycles in half of the rotaxanes to move past the blocking groups, thereby allowing the ions to de-thread (diffuse away from the macrocycle). In some cases, the half-life of the rotaxanes of the invention is at least double (e.g. at least three, four, five, six, seven, eight, nine or ten times longer than) the half-life of analogous molecules that differ from the rotaxanes only in that they comprise no blocking groups (i.e. analogous pseudo-rotaxanes, described below). The skilled person is aware of conditions that would promote the movement of the macrocycle past the blocking group(s), for example subjecting the rotaxane to high temperatures such as temperatures of 50 to 100° C.

In order to trap the macrocycle, the blocking groups, when components of the ion, necessarily extend outwards by a distance that, when measured relative to the centre of the cavity of the macrocycle, is greater than the internal radius of the macrocycle and thus prevent dissociation (unthreading) of the molecular strand from the macrocycle.

The distance by which the blocking group must extend thus depends on the size of the internal diameter of the macrocycle used. For example, the internal diameter of cucurbit[8]uril is about 0.88 nm, thus blocking groups capable of trapping cucurbit[8]uril extend at least about 0.44 nm (measured relative to the centre of the macrocycle). The internal diameter of cucurbit[10]uril is 1.13-1.24 nm. Thus, blocking groups capable of trapping cucurbit[10]uril extend at least 0.57-0.62 nm (measured relative to the centre of the macrocycle). For the avoidance of doubt, the blocking group when a component of the ion must extend relative to the centre of the cavity of the macrocycle by at least the length of the internal radius of the macrocycle. In other words, it is the radius of the ion comprising the blocking group (i.e. the length of the blocking group plus the other components of the ion, such as the Ar groups of L that extend from the centre of the cavity of the macrocycle). The blocking group itself need not be at least the length of the internal radius of the macrocycle. The skilled person is able to assess which blocking groups are capable of trapping which macrocycle.

The pseudo-rotaxanes disclosed herein comprise a macrocycle and an ion of formula [M_{z}L_{z1}]^{zn+}, with the proviso that each Ar of the ion is not substituted with one or more blocking groups capable of trapping the macrocycle. Subsequently, the macrocycle of the pseudo-rotaxanes disclosed herein is not locked in place by blocking groups, and the ion is able to de-thread (diffuse out of the macrocycle). Consequently, the DNA-binding properties of the ions comprised within the pseudo-rotaxanes is not “switched off”, i.e. the ions are able to diffuse out of the macrocycle and may bind to DNA.

As described above, viewed from a first aspect, there is provided a rotaxane comprising a macrocycle and an ion of formula [M_{z}L_{z1}]^{zn+}, wherein L is a ligand of formula (I):

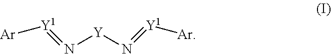

Y¹ of formula (I) is independently CR¹ or N. When Y¹ is N, the ligand of formula (I) comprises at least one azo group. In some embodiments, both Y¹ of formula (I) are CR¹ or both are N. In some embodiments, each Y¹ of formula (I) is CR¹.

R¹ is independently selected from H, C₁-C₄alkyl, amino, phenyl or C₃-C₅heteroaryl, wherein the phenyl or C₃-C₅heteroaryl is optionally substituted with any one or a combination selected from the group consisting of C₁-C₄alkoxy, C₁-C₄alkyleneC₁-C₄alkanoate, C₁-C₄alkanoate, hydroxy, C₁-C₄alkylol, carboxy, C₁-C₄alkanoate, C₁-C₄alkyl, halo, cyano, nitrite, C₁-C₄haloalkyl, C₁-C₄alkylthio, C₁-C₄alkyleneC₁-C₄alkylthio, C₁-C₄alkylsulfonate, diC₁-C₄alkylamino and C₁-C₄alkynyl.

For the avoidance of doubt, groups such as hydroxy, C₁-C₄alkylol, carboxy may be present in their deprotonated form.

In some embodiments, where each Y¹ is CR¹, each R¹ is the same.

In some embodiments, the phenyl or C₃-C₅heteroaryl is optionally substituted with any one or a combination selected from the group consisting of methoxy, methanoate, hydroxy, methylol, carboxy, methyl, halo, cyano, nitrite, halomethyl, methylthio, methylsulfonate and dimethylamino.

In some embodiments, the phenyl or C₃-C₅heteroaryl is not substituted.

In some embodiments, R¹ is independently selected from H, C₁-C₄alkyl and amino. In some embodiments, amino is a primary amino. In some embodiments, C₁-C₄alkyl is methyl or ethyl. In some embodiments, R¹ is independently selected from H, methyl and primary amino. In some embodiments, R¹ is H, thus Y¹ is CH.

Each Ar of formula (I) is positioned at either end of the ion of formula [M_{z}L_{z1}]^{zn+} and is independently a C₃-C₉heteroaryl comprising at least one nitrogen atom, is optionally substituted with one or more blocking groups capable of trapping the macrocycle, with the proviso that each end of the ion comprises at least one blocking group, and is optionally substituted with one or more substituents selected from the group consisting of hydroxy, C₁-C₄alkylol, carboxy, C₁-C₄alkanoate, C₁-C₄alkyl, C₁-C₄alkoxy, halo, cyano, nitrite, C₁-C₄haloalkyl, C₁-C₄alkylthio, C₁-C₄alkyleneC₁-C₄alkylthio, C₁-C₄alkylsulfonate, diC₁-C₄alkylamino and C₁-C₄alkynyl.

As shown in FIGS. 1 to 4 and 7, each Ar of formula (I) is positioned at either end of the ion of formula [M_{z}L_{z1}]^{zn+}; the ion of formula [M_{z}L_{z1}]^{zn+} forms a cylindrical-type shape with each Ar of formula (I) positioned at either end of the cylinder. Each Ar is optionally substituted with one or more blocking groups with the proviso that each end of the ion of formula [M_{z}L_{z1}]^{zn+} comprises at least one blocking group. For example, if the ion of formula [M_{z}L_{z1}]^{zn+} comprises three ligands of formula (I) (i.e. z1 is 3), two of the ligands may comprise no blocking groups (each Ar of the two ligands may not be substituted with one or more blocking groups) and one of the ligands may comprise one blocking group per Ar (each Ar of the one ligand may be substituted with one blocking group), resulting in each end of the ion of formula [M_{z}L₃]^{zn+} comprising one blocking group.

The number of blocking groups on each end of the cylinder need not be the same. For example, if the ion of formula [M_{z}L_{z1}]^{zn+} comprises three ligands of formula (I) (i.e. z1 is 3), one of the ligands may comprise no blocking groups (each Ar of the ligand may not be substituted with one or more blocking groups), one of the ligands may comprise one blocking group on one Ar and no blocking groups on the other Ar (only one of each Ar of the ligand may be substituted with one blocking group), and one of the ligands may comprise one blocking group per Ar (each Ar of the one ligand may be substituted with one blocking group). This results in one end of the ion of formula [M_{z}L₃]^{zn+} comprising one blocking group and the other end of the ion comprising two blocking groups.

In some embodiments, the ion of formula [M_{z}L_{z1}]^{zn+} comprises at least two blocking groups. In some embodiments, the ion of formula [M_{z}L_{z1}]^{zn+} comprises two to six blocking groups. For example, the ion of formula [M_{z}L_{z1}]^{zn+} may comprise three ligands of formula (I) (i.e. z1 may be 3) with each Ar of each ligand comprising 0 or 1 blocking group, with the proviso that each end of the ion comprises at least one blocking group. Thus, the ion of formula [M_{z}L₃]^{zn+} may comprise two to six blocking groups and when the ion of formula [M_{z}L₃]^{zn+} comprises two blocking groups one blocking group is situated at one end of the ion and the other is situated at the other end of the ion.

In some embodiments, each Ar is substituted with at least one blocking group.

In some embodiments the at least one nitrogen atom of the C₃-C₉heteroaryl is positioned 2 bonds away from the closest Y¹ of formula (I), as depicted in formula (IA):

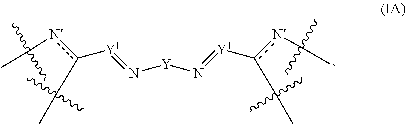

wherein N′ is N or NH, the dashed bonds are optionally present, and when present are part of a double bond, and the bonds cut by wavy lines lead to the rest of the C₃-C₉heteroaryl. Where the dashed bond is present, N′ is N.

In some embodiments, the optionally substituted C₃-C₉heteroaryl is independently an optionally substituted 2-imidazolyl, 5-imidazolyl, 2-pyridyl, 2-quinolinyl, 2-pyrazinyl, 1-isoquinolinyl, 3-isoquinolinyl, 2-pyrimidinyl, 4-pyrimidinyl, 2-pyrazinyl, 1,2,3-triazin-4-yl, 1,2,4-triazin-3-yl, 1,2,4-triazin-5-yl, 1,2,4-triazin-6-yl, 1,3,5-triazin-2-yl, 2-quinazolinyl, 4-quinazolinyl, 2-quinoxalinyl, 1,8-naphthyridin-2-yl, 1,7-naphthyridin-2-yl, 1,7-naphthyridin-6-yl, 1,7-naphthyridin-8-yl, 2,7-naphthyridin-1-yl, 2,7-naphthyridin-3-yl, 1,6-naphthyridin-2-yl, 1,6-naphthyridin-5-yl, 1,6-naphthyridin-7-yl1,5-naphthyridin-2-yl, 2,6-naphthyridin-1-yl, 2,6-naphthyridin-3-yl, 2-thiazolyl, 4-thiazolyl, 2-oxazolyl or 4-oxazolyl.

In some embodiments, the C₃-C₉heteroaryl is independently an optionally substituted 2-imidazolyl, 5-imidazolyl, 2-pyridyl, 2-quinolinyl, 2-pyrazinyl, 1-isoquinolinyl, 3-isoquinolinyl, 2-pyrimidinyl, 4-pyrimidinyl, 2-pyrazinyl, 1,2,3-triazin-4-yl, 1,2,4-triazin-3-yl, 1,2,4-triazin-5-yl, 1,2,4-triazin-6-yl, 1,3,5-triazin-2-yl, 2-quinazolinyl, 4-quinazolinyl, 2-quinoxalinyl, 1,8-naphthyridin-2-yl, 1,7-naphthyridin-2-yl, 1,7-naphthyridin-6-yl, 1,7-naphthyridin-8-yl, 2,7-naphthyridin-1-yl, 2,7-naphthyridin-3-yl, 1,6-naphthyridin-2-yl, 1,6-naphthyridin-5-yl, 1,6-naphthyridin-7-yl 1,5-naphthyridin-2-yl, 2,6-naphthyridin-1-yl or 2,6-naphthyridin-3-yl.

In some embodiments, the C₃-C₉heteroaryl is independently an optionally substituted 2-imidazolyl, 5-imidazolyl, 2-pyridyl, 2-quinolinyl, 2-pyrazinyl, 1-isoquinolinyl or 3-isoquinolinyl.

In some embodiments, the C₃-C₉heteroaryl is optionally substituted with one or more substituents selected from the group consisting of hydroxy, methylol, carboxy, methanoate, methyl, methoxy, halo, cyano, nitrite, halomethyl (such as trifluromethyl), methylthio, methylsulfonate and dimethylamino.

In some embodiments, the C₃-C₉heteroaryl is optionally substituted with any one or a combination selected from the group consisting of C₁-C₄alkanoate and C₁-C₄alkyl, such as methanoate and/or methyl.

In some embodiments, where z is 2 and z1 is 2, the C₃-C₉heteroaryl is optionally substituted, and where z is 2 and z1 is 3, the C₃-C₉heteroaryl is not substituted.

In some embodiments, the C₃-C₉heteroaryl is not substituted other than optional substitution with one or more blocking groups capable of trapping the macrocycle.

In some embodiments, the C₃-C₉heteroaryl is not substituted other than with at least one blocking group capable of trapping the macrocycle.

In some embodiments, each Ar of formula (I) is the same.

As described above, N—Y—N of formula (I) is N—N or is any one selected from the group consisting of (Ia) to (Ic):

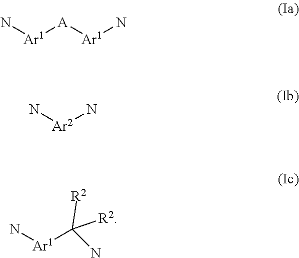

A of formula (Ia) is (CH₂)₁₋₄, O, NH, CHR², CR²₂, NR²S or SO₂. In some embodiments, A is (CH₂)₁₋₄, O, NH, CHR², CR²₂ or NR², such as CH₂.

Art is phenyl optionally substituted one or more times with R³, Ar² is phenyl or biphenyl optionally substituted one or more times with R³, and R² and R³ are independently any one or a combination selected from the group consisting of H, hydroxy, C₁-C₄alkylol, carboxy, C₁-C₄alkanoate, C₁-C₄alkyl, halo, cyano, nitrite, C₁-C₄haloalkyl, C₁-C₄alkylthio, C₁-C₄alkyleneC₁-C₄alkylthio, C₁-C₄alkylsulfonate, diC₁-C₄alkylamino and C₁-C₄alkynyl.

In some embodiments, R² and R³ are independently any one or a combination selected from the group consisting of H, hydroxy, methylol, carboxy, methanoate, methyl, halo, cyano, nitrite, halomethyl (such as trifluoromethyl), methylthio, methylsulfonate and dimethylamino.

In some embodiments, R² and R³ are independently H or C₁-C₄alkyl, such as methyl.

In some embodiments, Ar² is optionally substituted phenyl.

In some embodiments, N—Y—N is of formula (Ia).

As described above, each Ar is optionally substituted with one or more blocking groups capable of trapping the macrocycle of the rotaxane, by which is meant that the macrocycle is not able to move past the blocking groups, and is thus kinetically locked in place. As described above, the inventors have found that trapping the ions disclosed herein within a rotaxane switches off the biological activity of the ion. The biological activity may then be switched back on by removing blocking group(s) from the rotaxane, allowing the ion to de-thread (diffuse away from the macrocycle). Accordingly, in some embodiments, the blocking groups are capable of reversibly trapping the macrocycle.

Blocking groups may be removable by hydrogenation (typically catalytic hydrogenation). The removal of benzyl groups from the nitrogen atoms of imidazole groups is a well-established procedure (see Y. Yamamoto et aL, ACS Omega, 2020, 5, 6, 2699-2709), which may be applied when the blocking group is a benzyl derivative, such as a phenylC₁-C₄alkylene, or similar derivative such as a C₃-C₅heteroarylC₁-C₄alkylene. Catalytic hydrogenation reactions within cells have been reported (see, for example, J. J. Soldevila-Barreda et al., Nature Communications, 2015, 6, 6582; and J. J. Soldevila-Barreda and N. Metzler-Nolte, Chem. Rev., 2019, 119, 829-869), in which molecular or nanoparticular hydrogenation catalysts are used and are delivered to the cell, for example by an antibody.

Alternatively, blocking groups may be removable by hydrolysis (reaction with water, typically catalytic hydrolysis). The palladium catalysed hydrolysis of N-propargyl groups from N-propargyl-floxuridine and N-propargyloxycarbonyl groups from N-propargyloxycarbonyl-gemcitabine is reported by J. T. Weiss, N. O. Carragher and A. Unciti-Broceta in Scientific Reports, 2015, 5, 9329 and J. T. Weiss et al. in J. Med. Chem., 2014, 57, 5395-5404. The same method may be applied when the blocking group is a propargyl group or derivative (such as 1-(propyne-1,3-diyl)C₁-C₄alkyl of formula —CH₂CC(C₁-C₄alkyl)—see A, below, where R′ is C₁-C₄alkyl) or a propargyloxycarbonyl group or derivative (such as 2-propyn-1-yl formate—see B, below—and 3-C₁-C₄alkyl-2-propyn-1-yl formate (—C(O)OCH₂CC(C₁-C₄alkyl)—see C, below, where R′ is C₁-C₄alkyl).

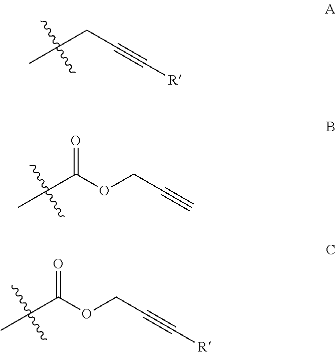

Palladium catalysts have also been reported to catalyse the removal of nitrogen-bound formate derivatives from drug molecules comprising amino groups (see, for example, T. L Bray et al., Chemical Science, 2018, 9, 7354-736). The same method may be applied when the blocking group is a formate derivative, such as benzyl formate (—C(O)OCH₂C₆H₅—see D, below) or propargyloxybenzyl formate (—C(O)OCH₂C₆H₄OCH₂CCH—see E, below).

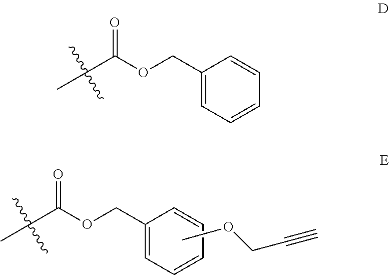

Blocking groups may be removable by reaction with a thiol. For example, imidazole-bound 2,4-dinitrophenyl groups are reported to be removable by reaction with thiols (see M. Beltrán, E. Pedroso and A. Grandas, Tetrahedron Letters, 1998, 39, 23, 4115-4118). The same method may be applied when the blocking group is a derivative of or similar to 2,4-dinitrophenyl. Since some cells are rich in thiols (notably glutathione), blocking groups removable by reaction with thiols could allow for targeted in-cell cleavage.

Alternatively, blocking groups may be removable by photo-irradiation. A number of photo-cleavable groups are known in the art, such as o-nitrobenzyl formate (notably 4,5-dimethoxy-2-nitrobenzyl formate—see F, below), o-nitrobenzyl (notably 4,5-dimethoxy-2-nitrobenzyl—see G, below), o-nitrobenzyl carbonate (notably 4,5-dimethoxy-2-nitrobenzyl carbonate—see H, below), phenacylcarboxylate (notably 2-(3,5-dimethoxyphenyI)-2-carboxy-1-phenylethanone) and coumarin-4-ethanoate derivatives.

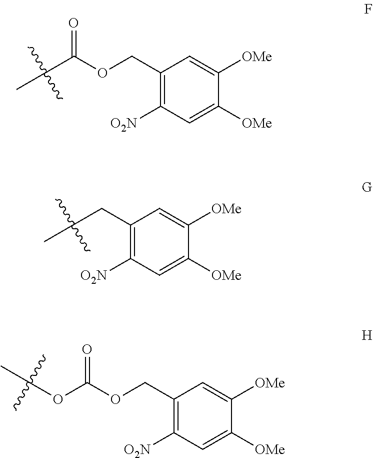 [SMILES: *C(=O)OCc1cc(OC)c(OC)cc1[N+](=O)[O-] ; *Cc1cc(OC)c(OC)cc1[N+](=O)[O-] ; *OC(=O)OCc1cc(OC)c(OC)cc1[N+](=O)[O-]]

For examples of photocleavable protection with nitrobenzyl derivatives, see S. R. Adams and R. Y. Tsien, Annu. Rev. Physiol., 1993, 55, 755-84 and K. Nakayama et al., Photochemistry and Photobiology, 2011, 87, 5, 1031-1035. The same photo-irradiation methods for removal of blocking groups may be applied when the blocking group is any one selected from the group consisting of 2-nitrophenyl formate, 2-nitrobenzyl, 2-nitrobenzyl carbonate, phenacylcarboxylate and coumarin-4-ethanoate, optionally substituted with one or more substitutents selected from the group consisting of C₁-C₄alkoxy, diC₁-C₄alkoxyphenyl, hydroxy, halo, amino, di(C₁-C₄alkyl)amino and carboxy.

Rather than remove blocking groups, a photo-induced isomerisation within the group (e.g. a blocking group comprising an azide) to switch the direction of the attached group (from sterically blocking to non-blocking) may allow for release of the macrocycle. Photo-cleavage or photo-switching might be used readily in near surface applications, or in deeper tissues with light introduced by optical fibres.

Blocking groups may be removable by enzymatic cleavage. Blocking groups may be cleaved by an endogenous enzyme that is highly expressed in the tissue of interest or in a specific target microbial. Examples of such enzymatic cleavage reactions include nitrophenylreductase-induced release of a nitrobenzyl group. This reaction has been exemplified in hypoxic tissues by I. N. Mistry et al. in Int. J Radiation Oncol. Biol. Phys., 2017, 98, 5, 1183e1196.

Alternatively, blocking groups may be removable by a non-endogenous enzyme that is carried to the tissue of interest by (for example) an antibody (in an ADEPT antibody-directed enzyme pro-drug therapy approach). For example, galactosidases may be used to cleave a sugar group (such as a β-galactoside or a glucuronide) or carboxypeptidases (such as bacterial carboxypeptidase (CPG2)) may be used to cleave a glutamate or a 2-aminoC₁-C₄alkyl-glutamate—see I, below.

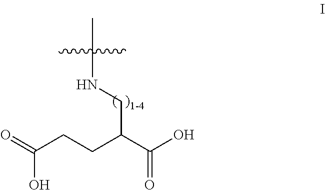 [SMILES: *NCC(CCC(=O)O)C(=O)O]

Self-immolative β-galactoside or glucuronide linkers are reported by I. Tranoy-Opalinski in European Journal of Medicinal Chemistry, 2014, 74, 302-313. By self-immolative is meant that the linker comprises a stable bond between the galactoside or glucuronide and the molecule that it is bound to, which becomes labile upon activation, leading to the rapid disassembly of the linker.

For examples of ADEPT strategies, see R. J. Francis et al., Br. J. Cancer., 2002, 87(6), 600-607; and I. Tranoy-Opalinski et al., European Journal of Medicinal Chemistry, 2014, 74, 302-313.

Accordingly, in some embodiments, blocking groups are removable by catalytic hydrogenation, reaction with a thiol, photo-irradiation, enzymatic cleavage, reduction or hydrolysis. In view of the knowledge in the field of functional groups that are removable by such methods, the skilled person is able to assess which blocking groups are removable by such methods.

Each blocking group may be the same or different. In some embodiments, each blocking group is the same.

In some embodiments, the blocking groups are any one or a combination selected from the group consisting of C₃-C₅heteroarylC₁-C₄alkylene, phenylC₁-C₄alkylene, propargyl, 1-(propyne-1,3-diyl)C₁-C₄alkyl, 2-propyn-1-yl formate, 3-C₁-C₄alkyl-2-propyn-1-yl formate, formate, C₁-C₄alkyl formate, benzyl formate, propargyloxybenzyl formate, 2-nitrophenyl formate, 2-nitrobenzyl 2-nitrobenzyl carbonate, phenacylcarboxylate, coumarin-4-ethanoate, dinitrophenyl (such as 2,4-dinitrophenyl), β-galactoside, glucuronide, glutamate, 2-aminoC₁-C₄alkyl-glutamate, wherein the 2-nitrophenyl formate, 2-nitrobenzyl, 2-nitrobenzyl carbonate, phenacylcarboxylate and coumarin-4-ethanoate are optionally substituted with one or more substitutents selected from the group consisting of C₁-C₄alkoxy, diC₁-C₄alkoxyphenyl, hydroxy, halo, amino and carboxy.

The C₃-C₉heteroarylC₁-C₄alkylene may comprise a pyridyl, imidazolyl, pyrazinyl, pyrimidinyl, 1,2,3-triazinyl, 1,2,4-triazinyl, 1,3,5-triazinyl or pyridazinyl. In some embodiments, the C₃-C₉heteroaryl comprises a pyridyl or imidazolyl, such as a pyridyl.

The C₃-C₉heteroarylC₁-C₄alkylene may comprise a methylene or ethylene, such as a methylene.

In some embodiments, the C₃-C₉heteroarylC₁-C₄alkylene is methylenepyridyl, such as 2-methylenepyridyl (picolyl).

The phenylC₁-C₄alkylene may be methylenephenyl (benzyl) or ethylenephenyl. In some embodiment, the phenylC₁-C₄alkylene is benzyl.

As described above, the 2-nitrophenyl formate, 2-nitrobenzyl, 2-nitrobenzyl carbonate, phenacylcarboxylate and coumarin-4-ethanoate are optionally substituted with one or more substitutents selected from the group consisting of C₁-C₄alkoxy, diC₁-C₄alkoxyphenyl, hydroxy, halo, amino, and carboxy. The C₁-C₄alkoxy may be methoxy, the diC₁-C₄alkoxyphenyl may be dimethoxyphenyl, the halo, may be fluoro, and the amino may be di(C₁-C₄alkyl)amino, such as dimethylamino. Thus, in some embodiments, the one or more optional substituents are selected from the group consisting of methoxy, dimethoxyphenyl, hydroxy, fluoro, dimethylamino and carboxy. The one or more optional substituents may be selected from the group consisting of C₁-C₄alkoxy, diC₁-C₄alkoxyphenyl and hydroxy.

When the blocking groups are 2-nitrophenyl formate, 2-nitrobenzyl or 2-nitrobenzyl carbonate, the phenyl may be additionally substituted, for example with methoxy groups, at the 4 and 5 positions.

When the blocking groups are phenacylcarboxylate, the methylene may be substituted with dimethoxyphenyl, for example with 3,5-dimethoxyphenyl.

In some embodiments, the blocking groups are any one or a combination selected from the group consisting of C₃-C₅heteroarylC₁-C₄alkylene, phenylC₁-C₄alkylene, propargyl, 2-propyn-1-yl formate, 2-nitrophenyl formate, 2-nitrobenzyl, 2-nitrobenzyl carbonate, phenacylcarboxylate, coumarin-4-ethanoate, dinitrophenyl, β-galactoside, glucuronide, glutamate, 2-aminoC₁-C₄alkyl-glutamate, wherein the 2-nitrophenyl formate, 2-nitrobenzyl, 2-nitrobenzyl carbonate, phenacylcarboxylate and coumarin-4-ethanoate are optionally substituted with one or more substitutents selected from the group consisting of C₁-C₄alkoxy, diC₁-C₄alkoxyphenyl, hydroxy, halo, amino and carboxy.

In some embodiments, the optionally substituted 2-nitrophenyl formate, is 4,5-dimethoxy-2-nitrophenyl formate, the optionally substituted 2-nitrobenzyl is 4,5-dimethoxy-2-nitrobenzyl, the optionally substituted 2-nitrobenzyl carbonate is 4,5-dimethoxy-2-nitrobenzyl carbonate and the optionally substituted phenacylcarboxylate is 2-(3,5-dimethoxyphenyI)-2-carboxy-1-phenylethanone.

In some embodiments, the blocking groups are C₃-C₅heteroarylC₁-C₄alkylene. In specific embodiments, the blocking groups are picolyl.

In some embodiments, L is of formula (IIa) or (IIb):

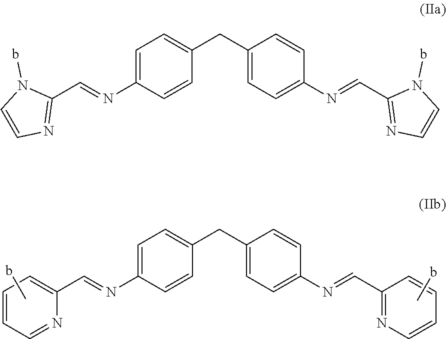

wherein each b is optionally present and is a blocking group, as defined above, with the proviso that each end of the ion of formula [M_{z}L_{z1}]^{zn+} of the rotaxane comprises at least one blocking group. In some embodiments, each b is present. In some embodiments, b, when present, is picolyl.

In some embodiments, L is of formula (IIa). In some embodiments, L is of formula (IIIa):

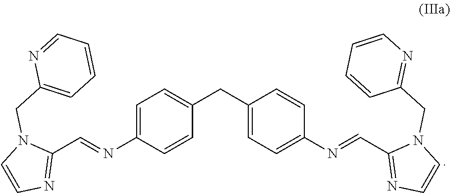 [SMILES: C(=N\c1ccc(Cc2ccc(/N=C/c3nccn3Cc3ccccn3)cc2)cc1)/c1nccn1Cc1ccccn1]

As described above, the rotaxanes disclosed herein comprise an ion of formula [M_{z}L_{z1}]^{zn+}, wherein M is a metal ion of oxidation state n⁺. M may be a metal ion of any one metal or a combination of metals selected from the group consisting of Fe, Ni, Ru, Co, Cu, Cd, Zn, Rh, Ir, Os, Pd, Pt, Ag and Mn. In some embodiments, M is a metal ion of any one metal or a combination of metals selected from the group consisting of Fe, Ni, Ru, Co, Cu, Cd, Zn, Rh, Ir, Os, Pd and Pt. In some embodiments, the ion of formula [M_{z}L_{z1}]^{zn+} does not comprise a combination of metals, i.e. it comprises only one type of metal. In specific embodiments, M is a metal ion of any one metal selected from the group consisting of Fe, Ni and Ru.

The oxidation state of the metal ion (n⁺) may be 1+ to 4+, such as 2+ or 3+. In some embodiments, n⁺ is 2⁺.

The number of metal ions M in formula [M_{z}L_{z1}]^{zn+} (z) may be 2 to 4. In some embodiments, z is 2 or 4. In specific embodiments, z is 2.

The number of ligands L in formula [M_{z}L_{z1}]^{zn+} (z1) may be 2 to 6. In some embodiments, z1 is 2, 3, 4 or 6, such as 2 or 3. In specific embodiments, z1 is 3.

The rotaxanes disclosed herein comprise a macrocycle. The macrocycle comprises a cavity in which an ion of formula [M_{z}L_{z1}]^{zn+} may reside. In some embodiments, the macrocycle is any one selected from the group consisting of a cucurbituril, crown ether, cyclodextrin, calixarene, pillararene, and metallo-organic macrocycle, or the macrocycle comprises a porphyrin, corrin, chlorin or aryl groups linked by C₁-C₄alkylenes, C₁-C₄ethers, C₂-C₄alkenylenes and C₂-C₄alkynylenes.

A cucurbituril is a macrocyclic molecule consisting of glycoluril monomers linked by methylene groups.

A crown ether is a macrocylic molecule derived from ethylene oxide. Crown ethers consist of ethyleneoxy repeating units, i.e., —CH₂CH₂O—.

A cyclodextrin is a macrocyclic molecule consisting of glucose subunits joined by α-1,4 glycosidic bonds.

A calixarene is defined herein as a macrocycle that is a hydroxyalkylation product of a phenol and an aldehyde (such as formaldehyde). The phenol and the aldehyde may be substituted, for example with C₁-C₄alkyl groups.

A metallo-organic macrocycle comprises aryl groups linked by metal ions alone or in combination with other organic linkers. For more information on metallo-organic macrocycles see: M. J. Prakash and M. S. Lah in Chem. Commun., 2009, 3326-3341 DOI: 10.1039/b902988e; Leininger, S.; Olenyuk B.; Stang, P. J. in Chem. Rev. 2000, 100, 853 doi.org/10.1021/cr9601324; M. Fujita in Chem. Soc. Rev., 1998, 27, 417-425 DOI: 10.1039/A827417Z; P.J. Altmann and A. Pöthig in J. Am. Chem. Soc. 2016, 138, 40, 13171-13174; Frischmann, P. D.; MacLachlan, M. J. in Chem. Soc. Rev. 2013, 42, 871.

A porphyrin is a heterocyclic macrocyclic compound composed of four modified pyrrole subunits interconnected at their a carbon atoms via methine bridges (═CH—). The pyrrole subunit may be modified by, for example, substitution with C₁-C₄alkyl, phenyl and/or C₂-C₄alkynyl groups.

A corrin is a heterocyclic macrocyclic compound composed of four modified pyrrole subunits, which are interconnected at their α carbon atoms via three methine bridges (═CH—), and one direct bond. The pyrrole subunit may be modified by, for example, substitution with C₁-C₄alkyl, phenyl and/or C₂-C₄alkynyl groups.

A chlorin defined herein is a heterocyclic macrocyclic compound that is an analogue of porphyrin, in which one pyrrole ring is replaced with a pyrroline ring, leading to a partial loss of aromaticity. The pyrrole or pryyoline subunits may be modified by, for example, substitution with C₁-C₄alkyl, phenyl and/or C₂-C₄alkynyl groups.

In some embodiments, the macrocycle is any one selected from the group consisting of a cucurbituril, crown ether, cyclodextrin, calixarene and pillararene. In specific embodiments, the macrocycle is any one selected from the group consisting of a cucurbituril, crown ether and cyclodextrin. In more specific embodiments, the macrocycle is a cucurbituril.

The size of macrocycle depends on the identity of the [M_{z}L_{z1}]^{zn+}. The macrocycle necessarily comprises a cavity in which an ion of formula [M_{z}L_{z1}]^{zn+} may reside. When the macrocycle is a curcubituril, it may comprise at least 8 monomers derived from glycoluril, such as 8 to 12 monomers. When the macrocycle is a crown ether derived from ethylene oxide, it may comprise at least 8 monomers derived from ethylene oxide, such as 8 to 12 monomers. When the macrocycle is a cyclodextrin, it may comprise at least 8 glucose subunits, such as 8 to 13 subunits. When the macrocycle is a calixarene, it may comprise at least 9 repeat units derived from a phenol and an aldehyde, such as 9 to 15 repeat units. When the macrocycle is a pillararene, it may comprise at least 9 hydroquinone or dialkoxybenzene units, such as 9 to 15 units. When the macrocycle is a metallo-organic macrocycle, it may contain a cavity with an internal diameter of at least 0.85 nm, such as 0.85 to 1.5 nm.

In specific embodiments, the macrocycle is a cucurbituril comprising 10 monomers, i.e. it is a cucurbit[10]uril.

In some specific embodiments, the rotaxane comprises a cucurbit[10]uril and an ion of formula [M₂L₃]⁴⁺, wherein L is a ligand of formula (IIIa) described above.

The zn⁺ charge of the ion of formula [M_{z}L_{z1}]^{zn+} may be balanced by one or more counterions. In some embodiments, the rotaxane comprises one or more counterions selected from the group consisting of chloride, hexafluorophosphate, bromide, iodide, nitrate, phosphate, sulphate, acetate, trifluoroacetate, propionate, glycolate, maleate, malonate, mesylate, fumarate, succinate, tartarate, citrate, benzoate and ascorbate. Typically, the one or more counterions are the same and balance the charge on the ion of formula [M_{z}L_{z1}]^{zn+}, i.e. the overall charge of the one or more counterions is zn⁻.

The rotaxanes of the invention may exist in different stereoisomeric forms. All stereoisomeric forms and mixtures thereof, including enantiomers and racemic mixtures, are included within the scope of the invention. Such stereoisomeric forms include enantiomers and diastereoisomers. Individual stereoisomers of compounds of the invention, i.e., associated with less than 5%, preferably less than 2% and in particular less than 1% of the other stereoisomer, are included. Mixtures of stereoisomers in any proportion, for example a racemic mixture comprising substantially equal amounts of two enantiomers are also included within the invention.

Solvates and isotopically-labelled rotaxanes are also included. Isotopically-labelled compounds are identical to those disclosed herein, but for the fact that one or more atoms are replaced by an atom having an atomic mass or mass number that is different from the atomic mass or mass number predominantly found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F and ³⁶Cl respectively.

All amorphous and crystalline forms of the rotaxanes are included.

Viewed from a second aspect, there is provided a method of synthesising a rotaxane, the method comprising:
(i) contacting a macrocycle and an ion of formula [M_{z}L_{z1}]^{zn+} to produce a pseudo-rotaxane, wherein the ion of formula [M_{z}L_{z1}]^{zn+} and the macrocycle is as defined in the first aspect, above, with the proviso that each Ar is not substituted with one or more blocking groups capable of trapping the macrocycle; and
(ii) reacting the pseudo-rotaxane with a compound of formula b-X to form the rotaxane, wherein b is a blocking group capable of trapping the macrocycle as defined in the first aspect above and X is a leaving group with the proviso that each end of the ion of formula [M_{z}L_{z1}]^{zn+} of the rotaxane comprises at least one blocking group. For the avoidance of doubt, and as described above, by a blocking group capable of trapping the macrocycle is meant that the movement of the macrocycle past the blocking group(s) is hindered, and the macrocycle is kinetically locked in place. The macrocycle need not be trapped by the blocking groups indefinitely.

In some embodiments, at least two equivalents of b-X with respect to the pseudo-rotaxane are employed. In some embodiments, two to six equivalents of b-X with respect to the pseudo-rotaxane are employed.

For the avoidance of doubt, the embodiments of the first aspect apply mutatis mutandis to this aspect. For example, the ion of formula [M_{z}L_{z1}]^{zn+} may be an ion of formula [M₂L₃]⁴⁺, wherein L is a ligand of formula (IIa), described above, without the blocking groups (b), the macrocycle may be cucurbit[10]uril, and b may be picolyl.

It will be understood that the contacting of the method may be achieved in a variety of ways. For example, the macrocycle may be added to a solvent to produce a solution or suspension comprising the macrocycle, to which the ion of formula [M_{z}L_{z1}]^{zn+} may be added as a solid or as part of a solution or suspension. Alternatively, the ion of formula [M_{z}L_{z1}]^{zn+} may be added to a solvent to produce a solution or suspension comprising the ion, to which the macrocycle may be added as a solid or as part of a solution or suspension. Otherwise, the macrocycle and ion of formula [M_{z}L_{z1}]^{zn+} may be combined as solids, to which a solvent may be added thereby forming a solution or suspension comprising both the macrocycle and ion.

Contacting the macrocycle and ion of formula [M_{z}L_{z1}]^{zn+} produces a pseudo-rotaxane. The pseudo-rotaxane is then reacted with a compound of formula b-X to form the rotaxane, wherein X is a leaving group. In some embodiments, X is any one selected from the group consisting of halo, C₁-C₄alkylsulfonate, C₁-C₄haloalkylsulfonate and phenylsulfonate optionally substituted one or more times with any one or a combination from the group consisting of C₁-C₄alkyl and C₁-C₄haloalkyl. X may be any one selected from the group consisting of halo, triflate, mesylate and tosylate, such as bromo, iodo, triflate, mesylate or tosylate. In some embodiments, X is bromo or iodo.

The reacting of the pseudo-rotaxane with b-X may be achieved in a variety of ways. For example, the pseudo-rotaxane may be added to a solvent to produce a solution or suspension comprising the pseudo-rotaxane, to which b-X may be added as a solid or as part of a solution or suspension. Alternatively, b-X may be added to a solvent to produce a solution or suspension comprising b-X, to which the pseudo-rotaxane may be added as a solid or as part of a solution or suspension. Otherwise, the pseudo-rotaxane and compound of formula b-X may be combined as solids, to which a solvent may be added thereby forming a solution or suspension comprising both the pseudo-rotaxane and compound of formula b-X.

The skilled person is aware of methods to improve contacting or reacting efficiency, such as agitating or stirring the components, increasing the temperature of the solvent or solution comprising the components, e.g. to temperatures of about 60° C. to about 100° C., such as about 70° C. to about 90° C., or about 80° C., and/or increasing the reaction time, e.g. to about 12 to about 24 hours. The skilled person is also aware of methods to work effectively with reagents that are unstable to air or water. In some embodiments, the contacting and/or reacting of the method is carried out in an inert atmosphere, such as under nitrogen or argon.

The contacting and reacting may take place in a one-pot synthesis, i.e. the pseudo-rotaxane may not be isolated before the reacting.

The solvent used in the method may be a protic solvent of high polarity, i.e. a protic solvent with a dielectric constant at 25° C. of ≥17, such as any one or a combination selected from the group consisting of methanol, water, ethanol, iso-propyl alcohol, n-propyl alcohol, n-butyl alcohol, and acetonitrile. In some embodiments, the solvent is methanol and/or water, such as methanol and water.

Following the reacting, the rotaxane may precipitate from the reaction mixture and may be isolated by filtration, e.g. vacuum filtration. The skilled person is aware of methods to encourage precipitation. For example, the volume of the reaction mixture may be reduced, e.g. by rotary evaporation, the temperature of the reaction mixture may be lowered, e.g. by refrigeration, and/or an anti-solvent may be used (in which the rotaxane is less soluble than the solvent already present in the reaction mixture). A suitable anti-solvent is miscible with the solvent already present in the reaction mixture. As the solvent and anti-solvent mix, precipitation of the rotaxane is encouraged. Alternatively, rotaxane may be isolated by removal of the solvent and washing away any impurities.

Viewed from a third aspect, there is provided a method of synthesising a rotaxane, the method comprising contacting a macrocycle and an ion of formula [M_{z}L_{z1}]^{zn+} at temperatures of about 50 to about 100° C., wherein the ion of formula [M_{z}L_{z1}]^{zn+} and the macrocycle is as defined in the first aspect, above.

For the avoidance of doubt, the embodiments of the first aspect apply mutatis mutandis to this aspect. For example, the ion of formula [M_{z}L_{z1}]^{zn+} may be an ion of formula [M₂L₃]⁴⁺, wherein L is a ligand of formula (IIIa), described above and the macrocycle may be cucurbit[10]uril.

It will be understood that the contacting of the method may be achieved in a variety of ways. For example, the macrocycle may be added to a solvent to produce a solution or suspension comprising the macrocycle, to which the ion of formula [M_{z}L_{z1}]^{zn+} may be added as a solid or as part of a solution or suspension. Alternatively, the ion of formula [M_{z}L_{z1}]^{zn+} may be added to a solvent to produce a solution or suspension comprising the ion, to which the macrocycle may be added as a solid or as part of a solution or suspension. Otherwise, the macrocycle and ion of formula [M_{z}L_{z1}]^{zn+} may be combined as solids, to which a solvent may be added thereby forming a solution or suspension comprising both the macrocycle and ion.

As described above, the skilled person is aware of methods to improve contacting efficiency, such as agitating or stirring the components and/or increasing the reaction time, e.g. to about 12 to about 24 hours. The skilled person is also aware of methods to work effectively with reagents that are unstable to air or water. In some embodiments, the contacting and/or reacting of the method is carried out in an inert atmosphere, such as under nitrogen or argon.

The solvent used and isolation of the rotaxane may be as described above in relation to the second aspect of the invention.

Viewed from a fourth aspect, there is provided a method of removing blocking group(s) from the rotaxane of the first aspect of the invention, the method comprising reacting the rotaxane with hydrogen, reacting the rotaxane with a thiol, photo-irradiating the rotaxane, contacting the rotaxane with an enzyme, reacting the rotaxane with a reducing agent or reacting the rotaxane with water.

For the avoidance of doubt, the method need not remove all of the blocking groups from the rotaxane. In some embodiments, the method removes the blocking group(s) from at least one end of the ion of formula [M_{z}L_{z1}]^{zn+} of the rotaxane. In some embodiments, the method removes at least one blocking group from the rotaxane. In some embodiments, the method removes at least two blocking groups from the rotaxane. In some embodiments, the method removes each blocking group from the rotaxane.

For the avoidance of doubt, the embodiments of the first aspect apply mutatis mutandis to the fourth aspect. For example, the ion of formula [M_{z}L_{z1}]^{zn+} may be an ion of formula [M₂L₃]⁴⁺, wherein L is a ligand of formula (IIIa), described above and the macrocycle may be cucurbit[10]uril.

Examples of the different methods that may be used to remove blocking groups from rotaxane are described in relation to the first aspect of the invention, above. In some embodiments, the reacting of the rotaxane with hydrogen is in the presence of a hydrogenation catalyst, such as palladium. The palladium may be in the form of molecular palladium or in the form of nanoparticles. Alternatively, the palladium may be incorporated into a polymer nanoparticle. In some embodiments, formic acid is used as a source of hydrogen. In some embodiments, the enzyme is any one selected from the group consisting of a nitrophenylreductase, galactosidase, carboxypeptidase, β-lactamase and β-glucuronidase. The carboxypeptidase may be a bacterial carboxypeptidase such as CPG2 and/or the β-glucuronidase may be human β-glucuronidase.

As described above, removing blocking groups from the rotaxane allows the ion of formula [M_{z}L_{z1}]^{zn+} to escape (diffuse out of) the macrocycle (de-rotaxanate) and bind to DNA and/or RNA (or other biological targets). Thus, removing blocking groups switches on the biological activity of the ion. De-rotaxanation of rotaxanes is exemplified by R. Barat et al. in Chem. Sci. 2015, 6, 2608-2613; A. Fernandes et al. in Angew. Chem. Int. Ed. 2009, 48, 6443 —6447; and A. Fernandes et al. in Chem. Commun. 2012, 48, 2083-2085. See also WO 2005/004795 (Univ. Cincinnati).

Viewed from a fifth aspect, there is provided an ion of formula [M_{z}L_{z1}]^{zn+}, as defined in the first aspect, wherein the blocking groups are capable of reversibly trapping a macrocycle. Such blocking groups are described in detail above, in relation to the first aspect. For the avoidance of doubt, the embodiments of the first aspect apply mutatis mutandis to the fifth aspect. For example, the ion of formula [M_{z}L_{z1}]^{zn+} may be an ion of formula [M₂L₃]⁴⁺, wherein L is a ligand of formula (IIIa), described above.

Whilst it is possible for the rotaxanes to be administered alone, it is typical to use a pharmaceutical formulation. Viewed from a sixth aspect, there is provided a pharmaceutical formulation comprising the rotaxane of the first aspect described above and one or more pharmaceutically acceptable excipients.

For the avoidance of doubt, the embodiments of the first aspect apply mutatis mutandis to the sixth aspect. For example, the ion of formula [M_{z}L_{z1}]^{zn+} may be an ion of formula [M₂L₃]⁴⁺, wherein L is a ligand of formula (IIIa), described above and the macrocycle may be cucurbit[10]uril.

The rotaxane (including salts, solvates or isotopically-labelled rotaxanes) within the formulation may be a solid, e.g. in a powder or crystalline form. Sometimes, to increase stability of the rotaxane, it may be freeze-dried or spray-dried before incorporation into the formulation. Freeze-drying a compound involves freezing the compound in the presence of solvent and separating the solvent from the compound by sublimation. Spray-drying a compound involves introducing a solution of the compound into an atomizer, which breaks up the solution into a spray of fine droplets. The droplets are ejected into a drying gas chamber where moisture vaporisation occurs, resulting in the formation of dry particles. Finally, the dried particles are separated from the drying medium.

The formulation may be a solid, e.g. in a powder or crystalline form. Alternatively, the formulation may be a solution or a suspension comprising a solvent, such as water.

The rotaxane (including salts, solvates or isotopically-labelled rotaxanes) or the formulation disclosed herein may be stored in any suitable container. The container may be adapted to prevent penetration of ultraviolet light. The container may be airtight and the rotaxane or formulation may be stored under an inert atmosphere, such as under nitrogen or argon. The rotaxane or formulation may be stored at room temperature, e.g. at about 20° C. Alternatively, it may be stored at temperatures lower than room temperature, e.g. in a refrigerator or freezer.

The excipient of the sixth aspect may be for identification or to make the rotaxane more appealing to the patient, for example by improving its taste, smell and/or appearance. The excipient may aid transport of the rotaxane to the site in the body where it is intended to act, for example by increasing the rate of dissolution of the rotaxane into the blood stream or by increasing the stability of the rotaxane, in order to increase its efficiency and prevent adverse side effects.

Excipients typically make up the bulk of pharmaceutical formulations and include diluents or fillers, binders, disintegrants, lubricants, colouring agents and preservatives. Diluents or fillers are inert ingredients. If the dosage of the compound of the invention is small then more diluents will be required to produce a formulation suitable for practical use. Conversely, if the dosage of the compound of the invention is high then fewer diluents will be required.

Binders are often used when the formulation is formed into a tablet. They add cohesiveness to powders in order to form granules. The binder allows the tablet to disintegrate upon ingestion so that the rotaxane dissolves. Disintegration of the formulation after administration may be facilitated through the use of a disintegrant.

An extensive overview of pharmaceutically acceptable excipients is described in the Handbook of Pharmaceutical Excipients, 6th Edition; Editors R. C. Rowe, P. J. Sheskey and M. E. Quinn, The Pharmaceutical Press, London, American Pharmacists Association, Washington, 2009. Any suitable pharmaceutically acceptable excipient is included within the sixth aspect.

Pharmaceutical formulations include those suitable for oral, nasal, parenteral (including subcutaneous, intravenous and intramuscular) or rectal administration. In some embodiments, the pharmaceutical formulation is suitable for parenteral administration, i.e. the pharmaceutical formulation is suitable for subcutaneous, intraveneous and/or intramuscular administration.

The pharmaceutical formulation may be compressed into solid dosage units, such as tablets, or be processed into capsules or suppositories. The pharmaceutical formulation may be injected, and may be prepared in the form of a solution, suspension or emulsion for injection. Alternatively, the pharmaceutical formulation may be administered as a spray, including a nasal or buccal spray. Otherwise, the pharmaceutical formulation may be administered as an implant or another preparation for immediate and/or sustained release. Typically, the pharmaceutical formulation is injected.

Viewed from a seventh aspect, there is provided a rotaxane of the first aspect or a pharmaceutical formulation of the sixth aspect for use as a medicament, for example for use in the treatment of any one or a combination selected from the group consisting of cancer, a viral disease and a bacterial disease.

For the avoidance of doubt, the embodiments of the first and sixth aspects apply mutatis mutandis to the sixth aspect. For example, the ion of formula [M_{z}L_{z1}]^{zn+} may be an ion of formula [M₂L₃]⁴⁺, wherein L is a ligand of formula (IIIa), described above, macrocycle may be cucurbit[10]uril, and the pharmaceutical formulation may be suitable for subcutaneous, intraveneous and/or intramuscular administration.

As described above, ions of formula [M_{z}L_{z1}]^{zn+} have been shown to bind to the heart of DNA and RNA 3-way junction structures, and other junctions and bulges, and arrest the proliferation of cancer cells and microbes including the HIV virus and coronavirus (severe acute respiratory syndrome coronavirus 2 (SARS-CoV-2)). See L. Cardo et al., Scientific Reports, 2018, 8, 13342; and A. D. Richards et al., Int. J. Antimicrob. Agents, 2009, 33, 469-472 for antimicrobial activity of the ions of the invention. See L. Cardo et al., Chem. Commun., 2011, 47 , 6575 — 6577; A. C. G. Hotze et al., Chemistry & Biology, 2008, 15, 1258-1267; and H. Qin et al., J. Am. Chem. Soc., 2017, 139, 45, 16201-16209 for anticancer activity of the ions of the invention.

Blocking group(s) of the rotaxane, when used as a medicament (such as for the treatment of cancer, a viral disease and/or a bacterial disease) may be removed from the rotaxane in situ. Blocking group(s) may be removed in situ by any of the methods described in relation to the first aspect of the invention. For example, blocking group(s) may be removed by reacting the rotaxane with hydrogen or a thiol, photo-irradiating the rotaxane, contacting the rotaxane with an enzyme, reacting the rotaxane with a reducing agent or reacting the rotaxane with water.

Viewed from an eighth aspect, there is provided a method of treatment, such as a method of treating cancer, a viral disease and/or a bacterial disease, comprising administering a therapeutically effective amount of a rotaxane of the first aspect or a pharmaceutical formulation of the fifth aspect to a subject.

For the avoidance of doubt, the embodiments of the first, sixth and seventh aspects apply mutatis mutandis to the eighth aspect. For example, the ion of formula [M_{z}L_{z1}]^{zn+} may be an ion of formula [M₂L₃]⁴⁺, wherein L is a ligand of formula (IIIa), described above, macrocycle may be cucurbit[10]uril, the pharmaceutical formulation may be suitable for subcutaneous, intraveneous and/or intramuscular administration, and blocking group(s) of the rotaxane may be removed from the rotaxane in situ.

The subject of the eighth aspect may be any animal, such as a mammal. The subject may be, and typically is, a human.

The subject may be suffering from or liable to suffer from cancer, a viral disease and/or a bacterial disease. Treatment of said subject comprises administering a therapeutically effective amount of rotaxane. The term “therapeutically effective amount” defines an amount of rotaxane that ameliorates the disease and produces the desired therapeutic or inhibitory effect. The skilled person is aware that an effective amount may vary depending on the particular rotaxane, the subject and the administration procedure. It is within the means and capacity of the skilled person to identify the therapeutically effective amount of rotaxane and pharmaceutical formulation comprising rotaxane via routine work and experimentation.

Any discussion herein of documents, acts, materials, devices, articles or the like is not to be taken as an admission that any or all of these matters form part of the prior art base or were common general knowledge in the field relevant to the present disclosure as it existed before the priority date of each claim of this application. It will be appreciated by those skilled in the art that numerous variations and/or modifications may be made to the invention as described herein without departing from the scope of the invention as described. The present embodiments are therefore to be considered for descriptive purposes and are not restrictive, and are not limited to the extent of that described in the embodiment. The person skilled in the art is to understand that the present embodiments may be read alone, or in combination, and may be combined with any one or a combination of the features described herein.

The subject-matter of each patent and non-patent literature reference cited herein is hereby incorporated by reference in its entirety.

The aspects and embodiments of this disclosure are further described in the following examples.

### EXAMPLES

Synthetic procedures suitable for the synthesis of a variety of ions of formula [M_{z}L_{z1}]^{zn+} are exemplified in WO 2005/033119 A1 (Univ. Warwick). For the avoidance of doubt, this patent application is incorporated by reference in its entirety.

### Materials and Methods

All starting materials and deuterated solvents were purchased from Aldrich, Fisher, Lancaster, Fluorochem and used without further purification. Electrospray Ionisation (ESI) analyses were performed in positive ionisation mode at the University of Birmingham on a Micromass LCT Time of Flight Mass Spectrometer or at Universite Aix-Marseille on a Waters SYNAPT G2 HDMS mass spectrometer or at Federal University of Sao Carlos on an Agilent LC-6545 Q-TOF-MS. UV/Visible spectra were recorded using a Cary 5000 Varian spectrophotometer using far UV grade solvents and quartz cuvettes. Microanalyses were conducted on Exeter Analytical CE44 CHN analyser by the University of Warwick Analytical Service and on CE Instruments EA1110 elemental analyzer by the University of Birmingham.

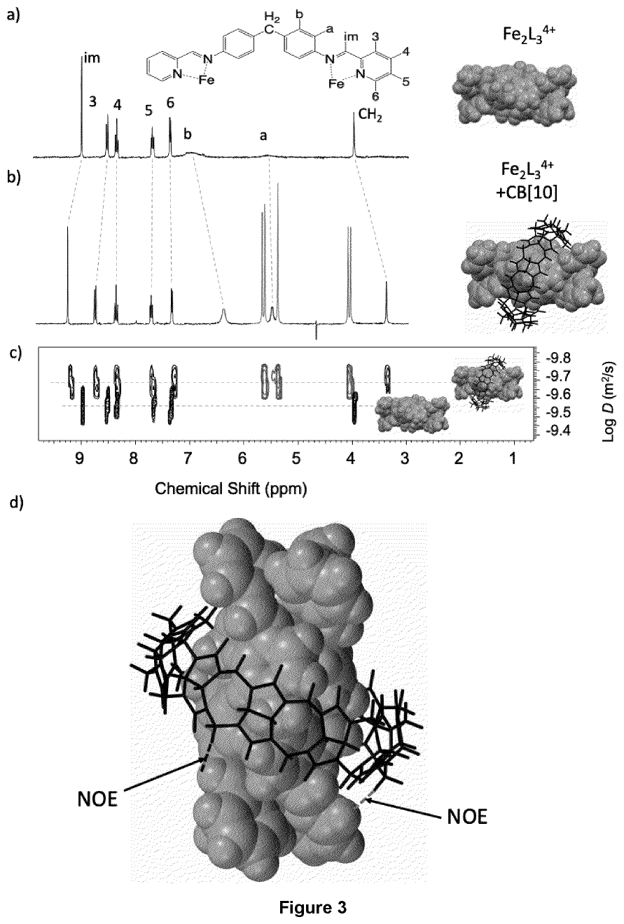

¹H and ¹³C NMR studies were carried out on ACIII-300, AVL-300 (300 MHz), AVIII-400 (400 MHz) Bruker spectrometers at Birmingham and Marseille, or using a Bruker AVANCE HD 500 MHz spectrometer equipped with a multi-nuclei 5 mm BBFO probe with Z-gradient using Bruker Topspin 3.5 PI7 software version at Marseille. See FIG. 3 for proton labelling system.

The COSY NMR sequence was a gradient sequence “cosygpprqf” with water suppression. The COSY spectrum was obtained with an F2 spectral width of 12 ppm and 2K data points and an F1 spectral width of 256 t1 increments and 32 scans, processed with SI2=2K and SI1=1k and pure cosine squared sine window functions applied to both dimensions.

The NOESY NMR sequence was a gradient sequence “noesygpphpr” with water suppression. The NOESY spectrum was obtained with an F2 spectral width of 15 ppm and 2K data points and an F1 spectral width of 256 t1 increments and 32 scans, processed with SI2=2K and SI1=1k and pure cosine squared sine window functions applied to both dimensions. Two NOESY spectra were recorded, the first with a mixing time=400 ms and the second with a mixing time=700 ms.

The ROESY NMR sequence was “roesyphpr” with water suppression. The ROESY spectrum was obtained with an F2 spectral width of 15 ppm and 2K data points and an F1 spectral width of 256 t1 increments and 56 scans for a mixing time=400 ms, processed with SI2=2K and SI1=1k and pure cosine squared sine window functions applied to both dimensions.

2D DOSY experiments were acquired using a pulse sequence that incorporated bipolar gradient pulses and a longitudinal eddy current delay (LED). A total of 16 gradient values were lineary sampled from 6% to 95%. 16 scans were acquired with 32k data points, for a total acquisition time of 165 min. The gradient pulse duration (d/2) and the diffusion time (D) were set to 1.7 ms and 100 ms, respectively, with a gradient recovery delay of 0.1 ms and a LED of 5 ms. The DOSY spectra were simply obtained by using the single-exponential fitting routine of the EDDOSY feature of the TopSpin software from Bruker Analytical Service.

### Synthesis

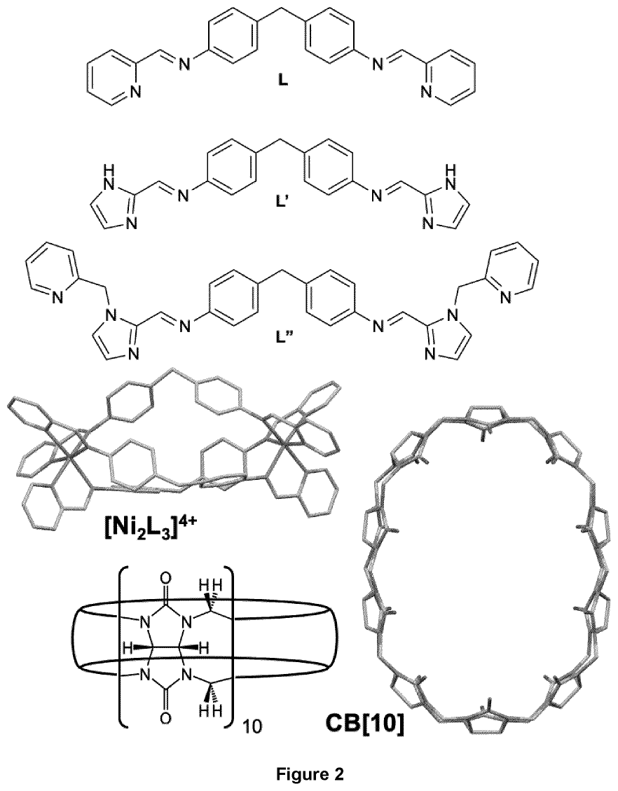

CB[10] (cucurbit[10]uril) was prepared as previously described (see S. M. Liu, P. Y. Zavalij and L. Isaacs, J. Am. Chem. Soc., 2005, 127, 16798-16799), as were ligand L (see L of FIG. 2) and the cylinders [M₂L₃][X]₄ (M=Fe, Ni, Ru; X=PF₆, CI) (see G. I. Pascu et al., Angew. Chem. Int. Ed., 2007, 46, 4374-4378; and M. J. Hannon et al., Chem. Commun., 1997, 1807-1808.

### Synthesis of Ligand L′ (see L′ of FIG. 2)

4,4′-methylenedianiline (0.790 g, 4.00 mmol) dissolved in methanol (15 mL) was added to 2-imidazolecarboxaldehdye (0.770 g, 8.00 mmol) in methanol (15 mL) giving a cloudy white solution which was stirred for 10 minutes under a nitrogen atmosphere. Glacial acetic acid (3 drops) was added and the solution heated under reflux for 2 hours. The solution was cooled to room temperature, and the white precipitate isolated by filtration. The precipitate was washed with methanol (2×10 mL) and dried in vacuo (1.25 g, 3.53 mmol, 88%).

¹H NMR: (400 MHz, DMSO-d₆) δ13.02 (NH, s, 1H), 8.40 (Him, s, 1H), 7.32 (H4/5, s, 1H), 7.30 (HPha/b, dt, J=8.6, 2.1 Hz, 2H), 7.25 (HPha/b, dt, J=8.6, 2.1 Hz, 2H), 7.18 (H4/5, s, 1 H), 3.98 (CH2, s, 1 H).

Mass spectrum (TOF MS EI+): m/z=355 [C₂₁H₁₈N₆+H]⁺.

### Synthesis of 2-Imidazole Nickel Complex ([Ni₂(L′)₃][X]₄, X=Cl, PF₆)

Hexafluorophosphate salt: The 2-imidazole ligand (L′, 0.11 g, 0.30 mmol) was suspended in methanol (10 mL) under a nitrogen atmosphere. A methanolic solution (10 mL) of nickel(II) chloride hexahydrate (0.05 g, 0.20 mmol) was added dropwise whilst stirring. After 45 minutes, excess ammonium hexafluorophosphate was added and the solution was immediately filtered through celite. The filtrate was chilled at 4° C. for two days. An orange precipitate was collected by vacuum filtration and washed with cold methanol (10 mL) before being dried in vacuo (0.05 g, 0.03 mmol, 28%).

¹H NMR: (300 MHz, Acetonitrile-d₃) δ74.77 (H5, br, 1H), 66.15 (Him, 1H), 25.16 (CH₂, 1H), 15.23 (HPha/b, 2H), −5.43 (HPha/b, br, 2H). The NH and H4 are expected to be very broad and are not unambiguously observed.

Mass spectrum (ES+): m/z=295 [Ni₂(C₂₁H₁₈N₆)₃]⁴⁺, 305 [Ni₂(C₂₁H₁₈N₆)₃+CH₃CN]⁴⁺, 316 [Ni₂(C₂₁H₁₈N₆)₃+2CH₃CN]⁴⁺, 326 [Ni₂(C₂₁H₁₈N₆)³⁺3CH₃CN]⁴⁺, 392 [Ni₂(C₂₁H₁₇N₆)₃−H]³⁺, 442 [Ni₂(C₂₁H₁₈N₆)₃(PF₆)]³⁺, 588 [Ni₂(C₂₁H₁₆N₆)₃−2H]²⁺, 735 [Ni₂(C₂₁ H₁₈N₆)₃(PF₆)₂]²⁺.

UV/VIS (acetonitrile): λₘₐₓ 323 nm/ε 56700 M⁻¹ cm⁻¹.

Chloride salt: The hexafluorophosphate salt was suspended in methanol (20 mL) and stirred for 30 minutes with Dowex (1×8 chloride form, 200-400 mesh). Dowex beads were removed by vacuum filtration and washed with methanol (10 mL). The solvent was removed from the filtrate under vacuo to yield an orange powder.

¹H NMR: (300 MHz, Methanol-d₄) δ74.2 (H5, br, 1H), 65.7 (Him, 1H), 26.1 (CH₂, 1H), 15.4 (HPha/b, 2H), −5.3 (HPha/b, br, 2H). The NH and H4 are expected to be very broad and are not unambiguously observed.

UV/VIS (acetonitrile): λₘₐₓ 321 nm/ε 72200 M⁻¹ cm⁻¹.

### Synthesis of [Ni₂(L″)₃[]PF₆]₄ by Post-Assembly Modification of [Ni₂(L′)₃][PF₆]₄ (See L″ of FIG. 2)

[Ni₂(L′)₃][PF₆]₄ (0.10 g, 0.06 mmol) and 2-(bromomethyl)pyridine hydrobromide (0.09 g, 0.34 mmol) were dissolved in acetonitrile (20 mL) under argon atmosphere. After 10 minutes, Hunig's base (0.18 mL, 1.02 mmol) was added and the solution was heated overnight under reflux. The solution was cooled to room temperature before being vacuum filtered and washed with acetonitrile (2 mL). The solvent was removed from the filtrate to give an orange, waxy crude product. The crude product was washed with chloroform (10 mL), the precipitate collected by vacuum filtration and washed with chloroform (2×5 mL) to yield an orange powder (0.05 g, 0.02 mmol, 35%).

¹H NMR: 300 MHz, Acetonitrile-d₃) δ78.8 (H5, 1H), 71.9 (Him, 1H), 24.4 (CH₂, 1 H), 15.23 (HPha/b, 2H), 8.54 (Hpy, 1 H), 7.79 (Hpy, 1 H), 7.51 (Hpy, 1 H), 7.20 (Hpy, 1 H), 4.20 (CH₂, s, 1 H), 1.43 (CH₂, s, 1 H), −4.6 (HPha/b, 2H). H4 is expected to be very broad and is not unambiguously observed.

Mass spectrum (TOF MS EI+): m/z=409 [Ni₂(C₃₃H₂₈N₈)₂(C₂₇H₂₃N₇)]⁴⁺, 432 [Ni₂(C₃₃H₂₈N₈)₃]⁴⁺, 545 [Ni₂(C₃₃H₂₈N₈)₂(C₂₇H₂₃N₇)−H]³⁺, 623 [Ni₂(C₃₃H₂₈N₈)₃(PF₆)₄]³⁺.

### Synthesis of [Ni₂(L″)₃][PF₆]₄ from the Pre-Alkylated Ligand L″

1-pyridine-2-ylmethyl-1H-Imidazole carboxaldehyde: N,N-diisopropylethylamine (3.0 mL, 17.01 mmol) was added to a stirring solution of 2-imidazolecarboxaldehyde (0.55 g, 5.67 mmol) and 2- (bromomethyl)pyridine hydrobromide (1.58 g, 6.24 mmol) in DMF (20 mL). The reaction mixture was heated at 80° C. overnight. After cooling to room temperature, the reaction was quenched with a saturated aqueous solution of NaHCO₃ (20 mL) and extracted with DCM (4×20 mL). The combined organic layers were washed with brine (30 mL), dried (MgSO₄), filtered, and concentrated under reduced pressure to afford brown oil. The crude mixture was purified by a Dionex Summit HPLC system using a Phenomenex C18 column (250 mm×21.2 mm) packed with Luna 10 μm on a solvent gradient starting from 0 to 33% of acetonitrile in water for 30 minutes to give the desired product as a yellow powder (0.13 g, 14%).

¹H NMR (CDCl₃, 300 MHz, 298 K): δ=9.82 (d, 1H, J=0.9 Hz, CHO), 8.55 (ddd, 1H, J=4.8, 1.7, 0.9 Hz, H11), 7.66 (td, 1H, J=7.7, 1.8 Hz, H9), 7.37 (br s, 1H, H4/5), 7.32 (1H, d, J=0.9 Hz, H4/5), 7.19-7.25 (m, 2H, H10, H8), 5.71 (5, 2H, H6).

¹³C NMR (CDCl₃, 400 MHz, 298 K): δ=180.3 (CHO), 153.3 (C7), 147.8 (C11), 141.2 (C2), 135.3 (C9), 130.0 (C4/5), 125.2 (C4/5), 121.3 (C10), 120.4 (C8), 50.4 (C6).

Mass spectrum (TOF MS EI+): m/z=187 [C₁₀H₉N₃O]⁺.

Melting point: 52-54° C.

Elemental analysis calculated (%) for C₁₀H₉N₃O: C, 64.2; H, 4.8; N, 22.0. Found (%): C, 64.1; H, 4.7; N, 22.0.

IR: 3103 (w), 2844 (w), 1672 (s), 1586 (m), 1569 (m), 1472 (m), 1412 (s), 1336 (m), 1303 (m), 1253 (m), 1287 (m), 1148 (m), 994 (m), 916 (m), 803 (m), 771 (5), 758 (s), 724 (m), 688 (m), 596 (m) cm⁻¹.

Alkylated ligand L″: An ethanolic solution of 4,4′-methylendianiline (15.4 mg, 0.077 mmol) was added dropwise to a solution of 1-pyridine-2-ylmethyl-1H-imidazole carboxaldehyde (29 mg, 0.155 mmol) in ethanol (5 mL). The reaction mixture was stirred at room temperature overnight. The off-white precipitate formed was collected by filtration, washed with cold ethanol and ether and dried in vacuo (25 mg, 59%).

¹H NMR (CDCl₃, 300 MHz, 298 K): δ=8.60 (ddd, 2H, J=4.8, 1.7, 0.9 Hz, Hpy6), 8.57 (5, 2H, Him), 7.62 (td, 2H, J=7.7, 1.8 Hz, Hpy4), 7.29 (H4/5 partially hidden by the solvent peak), 7.25 (br s, 2H, H4/5), 7.23-7.19 (m, 6H, Hpy5, HPh), 7.14-7.03 (m, 6H, Hpy3, HPh), 5.99 (s, 4H, CH2py), 4.00 (s, 2H, CH2).

Mass spectrum (TOF MS EI+): m/z=559 [C₃₃H₂₈N₈+Na]⁺

Elemental analysis calculated (%) for C₃₃H₂₈N₈: C, 73.9; H, 5.3; N, 20.9. Found (%): C, 73.6; H, 5.7; N, 21.2.

IR: 3094 (w), 3048 (w), 1625 (m), 1594 (m), 1570 (m), 1502 (m), 1467 (m), 1436 (m), 1279 (m), 1208 (m), 1106 (m), 1048 (m), 996 9m), 851 (m), 877 (s), 787 (m), 765 (s), 745 (s), 712 (m), 698 (m), 605 (m) cm⁻¹.

[Ni₂(L″)₃][Cl]₄: To a stirred solution of alkylated ligand L″ (18 mg, 0.034 mmol) in methanol (5 mL), nickel(II) chloride hexahydrate (5.3 mg, 0.022 mmol) in methanol (2 mL) was added and the mixture was refluxed overnight. After cooling to room temperature diethyl ether was added, the resulting orange precipitate was collected by filtration, washed with diethyl ether and dried in vacuo (12 mg, 57%). The chloride salt has excellent water solubility and was used for DNA binding studies and biological evaluation.

Mass spectrum (TOF MS EI+): m/z=432 [Ni₂(C₃₃H₂₈N₈)₃]⁴⁺, 588 [[Ni₂(C₃₃H₂₈N₈)₃]Cl]³⁺, 898 [[Ni₂(C₃₃H₂₈N₈)₃]Cl₂]2+.

Elemental analysis calculated (%) for [Ni₂(C₃₃H₂₈N₈)₃]Cl₄·8H₂O: C, 59.1; H, 5.0; N, 16.7.

Found (%): C, 59.5; H, 5.0; N, 16.8.

IR: 3344 (br), 3053 (w), 1592 (m), 1532 (w), 1484 (m), 1437 (s), 1331 (m), 1292 (m), 1210 (m), 1134 (m), 997 (w), 969 (m), 904 (m), 861 (w), 815 (w), 752 (s) cm⁻¹.

UV/VIS (water): λₘₐₓ/nm (ε/M⁻¹ cm⁻¹) 260 (32600), 328 (55600).

[Ni₂(L″)₃][PF₆]₄: Anion exchange with ammonium hexafluorophosphate in methanol afforded the corresponding hexafluorophosphate salt, yellow crystals of which, suitable for X-ray diffraction analysis, were obtained by slow diffusion of benzene into a solution of the complex in acetonitrile.

### Synthesis of Pseudo-Rotaxane CB10-[M₂L₃][Cl]₄

General procedure for the formation of the pseudo-rotaxane with parent cylinders, CB10-[M₂L₃][Cl]₄ (M=Fe, Ni or Ru): 1 equivalent of parent cylinder was dissolved in water and this solution (typically 1 mM) was employed to dissolve 0.9 equivalents of CB[10]. CB[10] appeared promptly soluble at room temperature and stirring was not needed. Using the same procedure, samples for NMR analysis (for Fe and Ru cylinders) were prepared using deuterated water (up to 10 mM solutions).

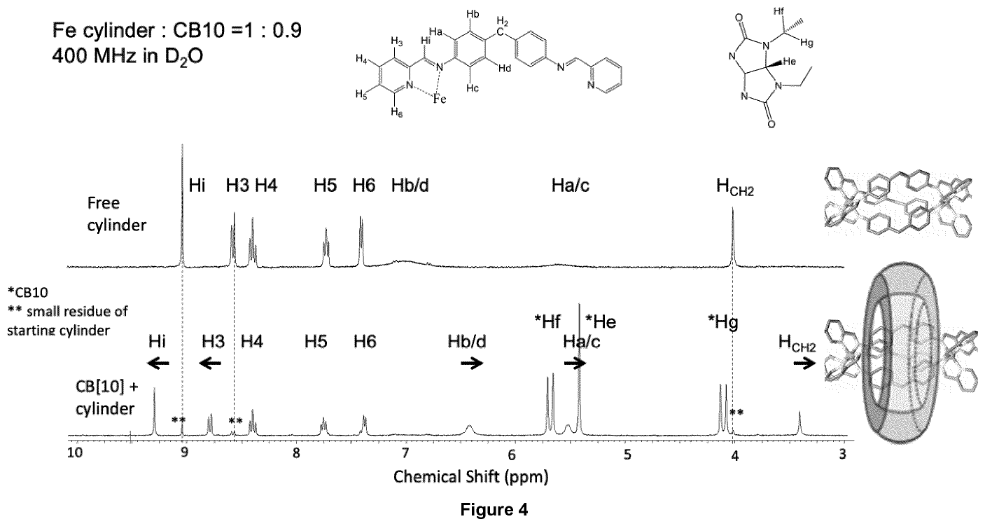

CB10-[Fe₂L₃][Cl]₄¹H NMR—see FIG. 4

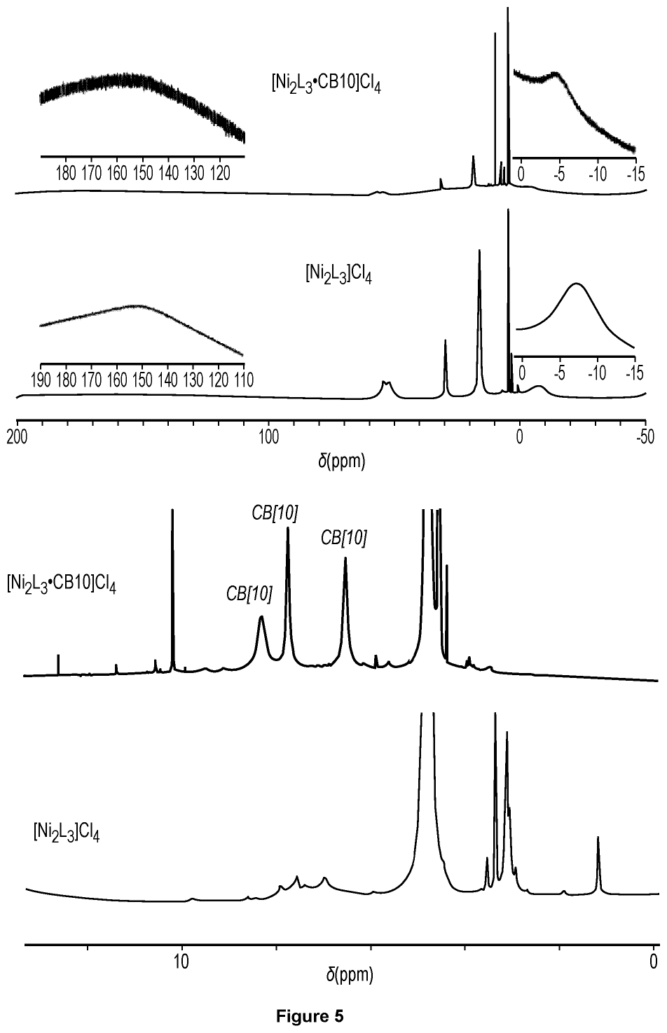

CB10-[Ni₂L₃][Cl]₄¹H NMR—see FIG. 5

CB10-[Ru₂L₃][Cl]₄¹H NMR (400 MHz, Deuterium Oxide) δ8.93 (Hi, s, 6H), 8.55 (H3, d, J=7.7 Hz, 6H), 8.20 (H4, td, J=7.8, 1.5 Hz, 6H), 7.70-7.48 (H5-H6 overlapping , m, 12H), 6.32 (Hb/d, d, J=7.7 Hz, 12H), 5.72-5.52 (Ha/c-Hf overlapping, m, 32H), 5.32 (He, s, 20H), 4.0 (Hg, d, J=15.1 Hz, 20H), 3.31 (HCH₂, s, 6H).

Mass spectrum (ESI) where L is the ligand with molecular formula C₂₅H₂₀N₄ and CB[10] is [C₆H₆N₄O₂]₁₀

CB10-[Fe₂L₃][Cl]₄ m/z=310.1 [(L)₃Fe₂]⁴⁺, 425.1 [(L)₃Fe₂Cl]³⁺, 725.5 [(L)₃Fe₂(CB10)]⁴⁺, 979.3 [(L)₃Fe₂Cl(CB10)]³⁺; Accurate mass measurement for the ion m/z 725.5: Calc 725.4670; Found 725.4672.

CB10-[Ni₂L₃][Cl]₄ m/z=m/z=311.1 [(L)₃Ni₂]⁴⁺, 427.1 [(L)₃Ni₂Cl]³⁺, 726.7 [(L)₃Ni₂(CB10)]⁴⁺, 981.0 [(L)₃Ni₂Cl(CB10)]³⁺; Accurate mass measurement for the ion m/z 726.7: Calc 726.7169; Found 726.7173.

CB10-[Ru₂L₃][Cl]₄ m/z=333.1 [(L)₃Ru₂]⁴⁺, 352.9 [(L)₃Ru₂K₂]⁴⁺, 528.8 [(L)₂Ru₂Cl₃]²⁺, 748.2 [(L)₃Ru₂(CB10)]⁴⁺

### Synthesis of Pseudo-Rotaxane CB10-[Ni₂L′₃][Cl]₄

[Ni₂L′₃][Cl]₄ (1.24 mg, 0.94 μmol) was dissolved in deuterated water (0.75 mL) and deuterated methanol (0.19mL). 0.9 equivalents of CB[10] (1.40 mg, 0.84 μmol) were added and the solution stirred until no more CB10 would dissolve. Excess CB[10] was removed by filtration.

Both the CB[10] bound cylinder and the free cylinder are observed in the NMR. Protons have been assigned CB- and cyl- for each species respectively.

¹H NMR: (300 MHz, Deuterium Oxide) δ74.81 (CB-H4 and cyl-H4, s, 12H), 25.93 (cyl-CH2, 5, 6H), 22.81 (CB-CH₂, s, 6H), 15.33 (cyl-HPha/b, s, 12H), 14.75 (CB-HPha/b s, 12H), 5.71 (HCB10-c, s, 20H), 5.41 (HCB10-a, s, 20H), 4.12 (HCB10-b, s, 20H), -4.88 (CB-HPha/b and cyl- HPha/b, 5, 24H).

Mass spectrum (ESI): m/z=355 [C₂₁ H₁₈N₆+H]⁺, 410 [Ni₂(C₂₁H₁₈N₆)Cl+H₂O]³⁺, 590 [Ni₂(C₂₁H₁₈N₆)₃−2H]²⁺, 711 [Ni₂(C₂₁ H₁₈N₆)₃+CB10]⁴⁺, 947 [Ni₂(C₂₁H₁₈N₆)³⁺+CB10-H]³⁺, 1420 [Ni₂(C₂₁H₁₈N₆)₃+CB10−2H]²⁺, 1426 [Ni₂(C₂₁H₁₈N₆)₃+CB10+H₂O−2H]²⁺.

### Synthesis of Rotaxane CB10-[Ni₂L″₃][Cl]₄

[Ni₂L′₃][Cl]₄ (0.04 g, 0.03 mmol) was dissolved in deionised water (10 mL) and methanol (0.3 mL) and added to CB[10] (0.05 g, 0.03 mmol) under an argon atmosphere. The solution was stirred for 20 minutes before 2-(bromomethyl)pyridine hydrobromide (0.05 g, 0.20 mmol) in deionised water (5 mL) was added. Hunig's base (0.09 mL, 0.51 mmol) was added to the solution after 10 minutes which was then heated to 80° C. and stirred overnight. The reaction was cooled to room temperature and quenched with saturated aqueous sodium hydrogen carbonate solution (15 mL). The solution was allowed to settle overnight before being filtered to remove a brown precipitate. The solvent was removed in vacuo and the resultant solid was suspended in methanol (25 mL). The precipitated salt was removed by vacuum filtration and the filtrate collected. The solvent was removed in vacuo and to yield a fine, cream precipitate (0.04 g, 0.11 mmol, 40%).

Mass spectrum (ESI): m/z=801 [Ni₂(C₃₃H₂₈N₈)(C₂₇H₂₃N₇)₂(CB10)]⁴⁺, 824 [Ni₂(C₃₃H₂₈N₈)₂(C₂₇H₂₃N₇)(CB10)]⁴⁺, 828 [Ni₂(C₃₃H₂₈N₈)₂(C₂₇H₂₃N₇)(CB10)+H₂O]⁴⁺, 847 [Ni₂(C₃₃H₂₈N₈)₃(CB10)]⁴⁺, 851 [Ni₂(C₃₃H₂₈N₈)₃(CB10)+H₂O]⁴⁺, 855 [Ni₂(C₃₃H₂₈N₈)₃(CB10)+2H₂O]⁴⁺, 1072 [Ni₂(C₃₃H₂₈N₈)(C₂₇H₂₃N₇)₂(CB10)+OH]³⁺, 1098 [Ni₂(C₃₃H₂₈N₈)₂(C₂₇H₂₃N₇)(CB10)−H]³⁺, 1104 [Ni₂(C₃₃H₂₈N₈)₂(C₂₇H₂₃N₇)(CB10)+OH]³⁺, 1134 [Ni₂(C₃₃H₂₈N₈)₃(CB10)+OH]³⁺. Accurate mass measurement for the ion m/z 847: Calc 847.2726; Found 847.2719.

An alternative procedure is as follows:

The nickel cylinder (0.025 g, 0.02 mmol) and CB10 (0.031 g, 0.02 mmol) were dissolved in water (15 mL) and allowed to stir under argon for 30 minutes to make a solution of pseudorotaxane. 2-(Bromomethyl)pyridine hydrobromide (0.030 g, 0.12 mmol) was separately dissolved in water (10 mL) under argon and cooled in an ice bath. Hunig's base (0.04 mL, 0.23 mmol) was added slowly to the 2-(Bromomethyl)pyridine to give a bright pink solution. The solution was added dropwise to the pseudorotaxane solution and water (5 mL) was used to make sure all the basic solution had been transferred. The reaction mixture was heated under reflux under argon for 16 hours at 100° C.

The solution was cooled to room temperature, water (30 mL) was added and the solution filtered under vacuo. The crude solution was reduced to dryness and the solid washed with chloroform (2×5 mL) before being allowed to dry. The remaining precipitate was dissolved in water (10 mL) and freeze dried overnight to yield a pale orange solid (0.042 g, 0.012 mmol, 63% yield).

### Alternative Method for Synthesis of Rotaxane [Ni₂L″₃·CB10][Cl]₄ Starting from [Ni₂L″₃][Cl]₄

[Ni₂L″₃][Cl]₄ (1.12 mg, 0.6 micromol) was dissolved in 80% H₂O 20% MeOH (1 ml) and treated with CB[10] (1.33 mg, 0.801 micromol). The solution was heated under reflux for 16 hours to afford a clear solution. The solution was centrifuged to remove any trace solids, the supernatant decanted, the solvent evaporated and the sample dried to obtain the product 0.96 mg (45% yield). The characterization data were analogous to those prepared by the previous method.

### Synthesis of Rotaxane [Fe₂L″₃·CB10][Cl]₄

Ligand L″ (4 mg, 0.0074 mmol, 3 equiv.) was suspended in 2 ml of degassed ethanol under argon and treated with a solution of FeCl₂·4H₂O (0.98 mg, 0.0049 mmol, 2 equiv.) in 3 ml of degassed ethanol. The mixture was stirred for 2 h at room temperature and the solvent then removed under reduced pressure. The solids were redissolved in 20% aqueous ethanol (5 ml) and solid CB[10] (4.12 mg, 0.00248 mmol, 1 equiv.) was added and the mixture stirred at 50° C. for 1 h. The solution was filtered through cotton wool and the solvent evaporated to obtain the product 7.7 mg (88% yield).

Mass Spec (ESI): m/z 845.8 [Fe₂(L″)₃·CB10]⁴⁺1139.3 {Fe₂(L″)₃·CB10+Cl}³⁺

### Synthesis of 1-(4,5-dimethoxynitrobenzyl)imidazole-2-carboxaldehyde

2-imidazole carboxaldehyde (0.06 g, 0.60 mmol) was placed under argon under dark conditions before dissolving in degassed acetonitrile (3 mL). Potassium carbonate (1.20 g, 0.17 mmol) dissolved in degassed water (3 mL) is added, and the solution heated to form a clear liquid. DMNB-bromide (0.17 g, 0.60 mmol) was dissolved in degassed acetonitrile (7 mL) under argon in the dark and added dropwise to the reaction solution. The reaction mixture was then refluxed at 80° C. overnight before cooling to room temperature and removing the solvent under vacuo. The precipitate was dissolved in dichloromethane (5 mL) and washed with brine (2×5 mL). The organic phase was collected and the solvent removed from the crude product which was then purified by silica column (10% ethyl acetate/chloroform and the 1% methanol/10% ethyl acetate/chloroform eluent) to yield the yellow oil product which crystallises at RT (45.6 mgs, 26% yield).

¹H NMR (400 MHz, Chloroform-d): δ9.84 (H_{aid}, d, J=0.9 Hz, 1H), 7.73 (H₈, s, 1H), 7.38 (H_{4/5}, d, =1.0 Hz, 1H), 7.28 (H_{4/5}, d, J=1.0 Hz, 1H), 6.29 (H₁₁, s, 1H), 5.01 (CH_{2(mid)}, s, 2H), 3.95 (CH₃, s, 3H), 3.75 (CH₃, s, 3H).

MS (ESI+, Solid): m/z=292 ([M+H]⁺).

### Synthesis of L^{dnmb}

4,4′-methylenedianiline (0.01 g, 0.06 mmol) was dissolved in degassed ethanol (5 mL) and added to a suspension of 1-(4,5-dimethoxy-2-nitrobenzyl)-2-imidazole carboxaldehyde (0.04 g, 0.14 mmol) in degassed ethanol (5 ml) under argon. Acetic acid (2 drops) was added and the solution was warmed at 50° C. for 2 hours before heating to 80° C. for 1 hour. The reaction solution was allowed to cool to room temperature overnight. The precipitate was collected by vacuum filtration through a 0.45 μm nylon filter and washed with small amounts of ethanol and cold methanol to give an off-white powder (0.04 g, 0.06 mmol, 95%).

¹H NMR (400 MHz, Chloroform-d) δ8.46 (Hᵢₘ, d, J=0.6 Hz, 1H), 7.63 (H₈, s, 1H), 7.26 (H_{4/5}, d, J=1.1 Hz, 1H), 7.19 (H₁₁, s, 1H), 7.11 (H_{4/5}, dd, J=1.1, 0.6 Hz, 1H), 7.09-7.03 (Ph_{a/b}, m, 2H), 6.93-6.88 (Ph_{a/b}, m, 2H), 6.12 (CH_{2(cap)}, d, J=2.1 Hz, 2H), 6.09 (CH_{2(central)}, s, 1 H), 3.86 (CH_{3(a/b)}, s, 3H), 3.58 (CH_{3(a/b)}, s, 3H).

MS (ESI+, (DCM)/MeCN): 745 ([C₃₉H₃₆N₈O₈+H]⁺), 767 ([C₃₉H₃₆N₈O₈+Na]⁺).

### Synthesis of DMNB capped Ni Cylinder [Ni₂L^{dmnb}₃][Cl]₄

L^{dnmb} (0.03 g, 0.03 mmol) was suspended in degassed methanol (1 mL) under inert and dark conditions. Nickel chloride hexahydrate (0.00(5) g, 0.02 mmol) was dissolved in degassed methanol (2 mL) and added slowly to the ligand suspension to stir overnight. The solution was filtered through a 0.45 μm filter before ammonium hexafluorophosphate (excess) was added to the filtrate and stirred for 1 hour. The solution was separated by vacuum filtration, washed with cold methanol (small amounts) and diethyl ether (small amounts) and the precipitate collected. The precipitate was then converted back to a chloride salt for analysis by Dowex conversion.

For conversions from hexafluorophosphate to chloride salts, Alfa Aesar Dowex beads were used (1×8, 200-400, Cl). The hexafluorophosphate complex is suspended in methanol and Dowex beads (excess) are added. The suspension is stirred at room temperature until the precipitate is dissolved and only the Dowex beads remain undissolved. The Dowex beads are removed by filtration and washed with methanol before the filtrate is collected and the solvent removed under vacuo. MS (ESI+, Methanol:Water): m/z=588 ([Ni₂(L″_{DMNB})₃]⁴⁺), 799 ([Ni₂(L″_{DMNB})₃+HCOO]³⁺. Alternatively, [Ni₂L′₃][Cl]₄ (0.03 g, 0.022 mmol) was dissolved in water (20 mL) and added to 4,5-dimethoxy-2-nitrobenzyl bromide (0.06 g, 0.20 mmol) under argon. Aqueous potassium carbonate (0.01 g in 1 mL) was added to the solution before heating under reflux for 13 hours. The cloudy solution was allowed to cool and concentrated under vacuo (to approximately 1 mL) before dissolving in methanol (40 mL). The solution was filtered through a 0.45 μm nylon filter to collect an orange filtrate. Ammonium hexafluorophosphate (excess) in methanol (minimal) was added and the light brown precipitate was collected by vacuum filtration.

MS (ESI+, MeCN): 588 ([Ni₂(L″_{DMNB})₃]⁴⁺, 795 ([Ni₂(L″_{DMNB})₃−H]³⁺.

### Synthesis of DMNB capped Ni rotaxane [Ni₂L^{dmnb}₃·CB10][Cl]₄

[Ni₂L^{dmnb}₃][Cl]₄ (0.005 g, 2 μmol) and CB[10] (0.004 g, 2 μmol) were suspended in methanol (1 mL) and water (4.5 mL) and heated to 60° C. overnight. The solution was cooled to room temperature, filtered and washed with cold methanol (small amounts). The solvent was removed from the filtrate by freeze drying.

MS (ESI+, Methanol:Water): m/z=1003 ([Ni₂(L″_{DMNB})₃+CB10]⁴⁺).

Alternatively, [Ni₂L′₃][Cl]₄ (0.02 g, 0.015 mmol) and CB[10] (0.025 g, 0.015 mmol) were stirred in water (15 mL) under argon until a clear solution was observed. This pseudorotaxane solution was then added to the DMNB-bromide (0.038 g, 0.136 mmol, 9 equivalents) under argon in water (5 mL) before aqueous potassium carbonate solution (0.3 mL, 20 mg/mL) was added. The reaction solution was then heated under reflux overnight at 100° C. The solution was cooled to room temperature and filtered through a 0.45 μrn nylon filter and the filtrate treated with methanolic ammonium hexafluorophosphate (excess) to precipitate the hexafluorophosphate salt of the complex at 4° C. overnight. The dark red precipitate was washed with cold methanol (small amounts) then suspended in methanol (10 mL) with Dowex beads and stirred until the precipitate had dissolved. The Dowex beads were removed by vacuum filtration and the solvent removed from the filtrate under vacuo to give a green precipitate of the corresponding chloride salt (0.008 g from 25% of reagent solution, 72% yield).

MS (ESI): m/z=1003 ([Ni₂(L″_{DMNB})₃+CB10]⁴⁺), 1364 ([Ni₂(L″_{DMNB})₃+CB10]³⁺).

### Synthesis of DNB Aldehyde

2-imidazole carboxaldehyde (103 mg, 1.07 mmol) was dissolved in acetonitrile (20 mL). To this solution was added potassium carbonate (223 mg, 1.61 mmol), followed by dropwise addition of an acetonitrile solution (20 mL) of 1-fluoro-2,4-dinitrobenzene (200 mg, 1.07 mmol). The mixture was heated under reflux for 67 hours under argon.

The mixture was filtered through cotton wool, concentrated in vacuo and the product purified by column chromatography on silica eluting with 88% chloroform, 10% ethyl acetate, 2% methanol. Yield 196 mg (75%) ¹H NMR (400 MHz, Acetonitrile-d3) δ9.68 (d, J=0.9 Hz, 1 H, CHO), 8.96 (dd, J=2.6, 0.4 Hz, 1 H, DNB-H), 8.64 (dd, J=8.7, 2.6 Hz, 1 H, DNB-H), 7.83 (dd, J=8.7, 0.4 Hz, 1 H, DNB-H), 7.54 (t, J=1.0 Hz, 1 H, Imidazole-H), 7.52 (d, J=1.1 Hz, 1H, Imidazole-H). Mass spectrum (ESI): m/z=263.04 (C₁₀H₇N₄O₅) [M+H]⁺

### Synthesis of DNB Capped Ligand (L^{dnb})

DNB aldehyde (50 mg, 0.19 mmol, 2.1 equivalents) and 4,4′ Methylene dianiline (18 mg, 0.09 mmol, 1 equivalent) and 15 ml ethanol were added to a 25 ml round bottom flask and stirred at room temperature over night to obtain the ligand as a yellow precipitate. The precipitate was collected by filtration, washed with cold ethanol and dried over vacuum. Yield 50 mg (80%). ¹H NMR (400 MHz, DMSO-d6) δ8.92 (d, J=2.6 Hz, 1 H, DNB-Ha), 8.67 (dd, J=8.7, 2.6 Hz, 1 H, DNB-Hb), 8.47 (d, J=0.6 Hz, 1 H, Imine-H), 7.98 (d, J=8.6 Hz, 1 H, DNB-Hc), 7.75 (dd, J=1.2, 0.6 Hz, 1 H, Imidazole-H), 7.47 (d, J=1.2 Hz, 1H, Imidazole-H), 7.14 (d, J=8.4 Hz, 2H, Ph-2H), 6.93 (d, J=8.4 Hz, 2H, Ph-2H), 3.86(s, 2H, CH2). Mass spectrum (ESI): m/z=686.4

### Synthesis of DNB Capped Ni Cylinder [Ni₂(L^{dnb})₃]Cl₄

L^{dnb} (20 mg, 0.029 mmol, 3 equivalents) was suspended in 5 ml dry methanol under argon. NiCl₂·6H₂O (4.62 mg, 0.019 mmol) in 5 ml ethanol was slowly added to the suspension. The reaction mixture was heated to 50° C. for 1 h. The solution became transparent yellow. The reaction mixture was filtered and excess methanolic NH₄PF₆ was added to the filtrate. The precipitate was collected by filtration and washed with methanol, and then converted back to the chloride salt using the dowex procedures outlined above to afford the product. Yield 18 mg (80%). Mass spectrum (ESI): m/z=544.0949 ([Ni₂(L^{dnb})₃]⁴⁺)

### Synthesis of DNB Capped Rotaxane [Ni₂(L^{dnb})₃·CB10]Cl₄

A suspension of CB[10] (10 mg, 0.006 mmol, 1.1 equivalent) was added to a degassed aqueous (10 ml) solution of [Ni₂(L′)₃]Cl₄ (7.2 mg, 0.0055 mmol, 1 equivalent). The mixture was stirred under argon for 10 minutes. To the mixture was added aqueous (5 mL) potassium carbonate (9.2 mg, 0.066 mmol, 12 equivalent) followed by aqueous (5 mL) 1-fluoro-2,4-dinitrobenzene (18.6 mg, 0.01 mmol, 18 equivalent), and the mixture was heated to 80° C. for 60 minutes. The yellow/orange precipitate was collected by filtration and washed with water, methanol, ether, and chloroform and dried under high vacuum to yield 16.5 mg (yield 76%). Mass spectrum (ESI): m/z=959.47 ([Ni₂(L^{dnb})₃·CB10]⁴⁺).

### Biophysics Experimental

Ultrapure water was used throughout all biophysical work. For Circular and Linear dichroism, DNA samples were made up from Calf thymus DNA sodium salt (sigma Aldrich) by dissolving in milli-Q water and washed using a 10 kda MWCO centrifuge tube. The solution was then quantified by UV-Vis spectroscopy (Cary 5000 NIR spectrometer) by ε258=13200 mol−1 dm3 cm−1 to give a concentration in DNA base pairs. This stock solution was kept frozen with fresh aliquot being taken out for each experiment. Fresh buffer was made up before each experiment (see experimental for specifics). Complexes were dissolved in ultrapure H2O only with fresh solutions being used for each batch of experiments.

Circular dichroism (CD) spectra were recorded on a Chirascan+ spectrometer (Applied Photophysics limited). The samples were scanned in a 1 cm path length cuvette between 800 and 200 nm with 3 repeats at 1 nm step size and 0.5 s dwell time per point. Titrations were carried out at a constant concentration of DNA, sodium chloride (10 mM) and sodium cacodylate buffer (1 mM, pH 7.3) by adding compensating solutions of 2× DNA/Buffer of equal volume to the titre of the complex solution. The concentration of complex in the cuvette was increased step wise by adding set volumes of a stock complex solution. The R value refers to the ratio of DNA base pairs to complex, i.e. R60=60 bp for every 1 complex, R4=4 bp per complex. Flow Linear Dichroism (LD) was carried out on the same Chirascan+ spectrometer (Applied Photophysics limited) using the LD accessory (Applied Photophysics limited). The LD cell has an angular gap of 0.25 mm giving an overall path length of 0.5 mm. Samples volumes began at 175 μl and stopped at 250 μl. The cell was rotated at 40 revolution's per second to optimise DNA signal. The titration series was carried out the same as in the CD studies, with a 3-minute incubation time at a lower revolution speed of 4 revolution's per second.

Gel electrophoresis studies were performed using following oligonucleotide sequences (from Eurofins Germany): 5′-CGGAACGGCACTCG-3′ (S1), 5′-CGAGTGCAGCGTGG-3′ (S2), 5′-CCACGCTCGTTCCG-3′ (S3). 1.2 pL of S1 was radiolabelled using 1 μL of adenosine triphosphate γ 32P (Perkin Elmer) at the 5′ end using 2 μL of T4 bacteriophage polynucleotide kinase (New England Biolabs) by incubating them at 37 degrees Celsius for 1 hr in 2 μL of 10×PNK buffer (New England Biolabs) made up to 20 μL with nuclease free water (ThermoFisher Scientific). This solution was heated to 80° C. for 3 minutes to inactivate the enzyme and before being purified using a QAIquick nucleotide removal column (qiagen), this was washed twice after binding to the column, and then eluted with 60 μL of nuclease free water to leave a 4 μM solution of radio-labelled S1*. PAGE Polyacrylamide gels were prepared by mixing 25 ml of 29:1 acrylamide/bis-acrylamide (National Diagnostic, protogel) with 5 mL of 10× Tris-Boric acid buffer (890 mM each, pH 8.3) and 20 mL of Milli-Q water. To this 400 μL of a 10% w/v ammonium persulfate/water solution and 40 μL of TEMED were added to initialise polymerisation. This is then immediately poured between 2 glass plates and a comb inserted at the top this is then left to set for 1 hr. Samples are 10 μL, prepared in Tris base and boric acid 89 mM and NaCI 10 Mm, with the oligomers Si*, S2 and S3 combined first before addition of complex. The samples are mixed carefully and incubated for 1 hr at 37° C., followed by centrifugation and addition of 5 μL of 30% w/v of glycerol in water (total volume 15 μl). The gel is placed in the electrophoresis apparatus (ThermoFisher Scientific) with 1× TB buffer and samples were loaded into the wells. Gels were run at 140 V for 2 hours, gently removed from the plates and placed in a cassette with a phosphor imaging screen and exposed for up to 2 hours. The phosphor screens were then imaged on a BIO-RAD FarosFX Plus molecular imager.

### Cell Biology Studies

General: All materials were purchased from Sigma Aldrich or Thermofisher Scientific whilst cell lines were purchased from the Health Protection Agency, European collection of cell cultures (ECACC). All cell culture experiments were performed with sterile consumables and solutions. Cell biology experiments were performed using A2780 and MDA-MB-231 cell lines cultured in RPMI 1640 and DMEM media respectively (both previously supplemented with 10% FBS, 1% L-glutamine and 1 Penicillin/Streptomycin), at 37° C. and humidified atmosphere (5% CO₂, 95% air). Cells were refreshed every 2 days and regularly checked for the absence of contamination. 1 mM stock solutions of complex or complex-CB10 systems were prepared in sterile water and concentrations checked by UV-Vis.

Cell viability assays: 96 well plates were seeded overnight with defined number of cells (8000 and 10000 cells in respective 100 μL of media for A2780 and MDA-MB-231 respectively). Cells were treated with different concentration of cylinders or cylinder-CB10 systems (typically 1, 2.5, 5, 10, 25, 50, 75, 140 μM in respective media) for 72 h in an incubator (37° C., 5% CO2, 95% air), followed by washing PBS (3×) and treatment with 3-(4,5-dimethylthiazolyl-2)-2, 5-diphenyltetrazolium bromide (MTT, final concentration 0.5 mg/mL) for 2 hours. The media was carefully removed from each well, the purple crystals of formazan dissolved in 200 μL DMSO and absorbance at 550 nm was measured using a microplate reader (Infinite 200 Pro, Tecan). Cell viability curves and statistic data (using two-way ANOVA) were generated with GraphPad employing measurements from at least three independent repeats of triplicated experiments.

In addition to the IC₅₀ values described below, the following were also obtained:
[Ni₂L′₃]⁴⁺ IC₅₀, 72 h, MDA-MB-231 163±10 μM
[Ni₂L′₃]⁴⁺ IC₅₀, 72 h, A2780cis 83±6 μM

Cell Uptake by ICP-MS: MDA-MB-231 cells (106 in 10 mL medium previously seeded overnight in T-75 flasks) were incubated with 50 μM of compound (complexes or complex-CB10 systems only containing Ru and Ni). After 24 hours medium was removed, cells washed 3× with PBS and detached by treatment with trypsin. The suspensions were centrifuged at 300 g, cell pellets re-suspended in PBS and counted. Suspensions were centrifuged again and pellets were kept at −80° C. until needed for digestion and ICP-MS analysis. One day before analysis, pellets were digested by treating with 300 μL of concentrated nitric acid (60-70%) overnight at 80° C. Each sample was diluted with water (TraceSELECT grade), to reduce nitric acid concentration up to ˜3%. Samples were analysed the same day by ICP-MS spectrometry (Agilent LC-ICP-MS, 7500cx) at the Mass Spectrometry facility of the University of Warwick, UK.

### X-Ray Crystallography

Suitable crystals were selected and datasets for [Ni₂L″₃][PF₆]₄ and [Fe₂L″₃][PF₆]₄ were measured on a Bruker APEXII CCD diffractometer at the window of a Bruker FR591 rotating anode (λ_{Mo-Kα}=0.71073 Å) by the National Crystallography Service. The data collections were driven by COLLECT and processed by DENZO. Absorption corrections were applied using SADABS. The structures were solved using SheIXT and were refined by a full-matrix least-squares procedure on F2 in SheIXL. Figures and reports were produced using OLEX2. All non-hydrogen atoms were refined with anisotropic displacement parameters. All hydrogen atoms were added at calculated positions and refined by use of a riding model with isotropic displacement parameters based on the equivalent isotropic displacement parameter (Ueq) of the parent atom. CCDC 1989563 - CCDC 1989564 contain the crystallographic data. These data can be obtained free of charge from The Cambridge Crystallographic Data Centre via www.ccdc.cam.ac.uk/data request/cif.

Crystal structure determination of [Ni₂L″₃][PF₆]₄: C₁₁₁H₉₆F₂₄N₂₄Ni₂P₄ (M=2463.41 g/mol): triclinic, space group P-1 (no. 2), a=13.3692(9) Å, b=22.2187(16) Å, c=22.4798(18) Å, α=113.514(3)°, β=102.465(3)°, γ=97.137(4)°, V=5811.4(8) Å3, Z=2, T=120(2) K, μ(MoKα)=0.476 mm−1, Dcalc=1.408 g/cm3, 68734 reflections measured (5.784°≤2θ≤50.054°), 20086 unique (Rint=0.3200, Rsigma=0.4293) which were used in all calculations. The final R1 was 0.1942 (I>2σ(I)) and wR2 was 0.3900 (all data).

The structure incorporates seven voids of total volume 529 Å3. These are filled with solvent molecules, but they are so highly disordered that it was not possible to locate them in the electron density and/or successfully refine them. For this reason, the SQUEEZE routine (Spek, 2009) was used to remove the contribution of the electron density of the disordered solvent molecules from the diffraction data, and the structure was then refined with only the nickel complex, the PF₆ anions and two molecules of solvent benzene present. The highly disordered solvent molecules resulted in very weak diffraction data, especially at high angles and this led to rather high agreement statistics.

Crystal structure determination of [Fe₂L″₃][PF₆]₄: C_{108.5}H_{94.5}F₂₄Fe₂N₂₅P₄ (M=2440.17 g/mol): triclinic, space group P-1 (no. 2), a=14.6717(4) Å, b=18.6418(5) Å, c=22.0009(4) Å, α=66.665(2)°, β=83.538(2)°, γ=80.3500(10)°, V=5439.9(2) Å3, Z=2, T=120(2) K, μ(MoKα)=0.428 mm−1, Dcalc=1.490 g/cm3, 74537 reflections measured (5.824°≤2θ≤50.054°), 19136 unique (Rint=0.0847, Rsigma=0.1000) which were used in all calculations. The final R1 was 0.1041 (I>2σ(I)) and wR2 was 0.2129 (all data).

In the iron(II) complex the pyridylmethyl group C(95)-C(99), N(24)/C(95A)-C(99A), N(24A) is disordered over two sites at a percentage occupancy ratio of 70.4 (8):29.6 (8). The structure contains four PF₆ anions per di-iron(II) complex, an acetonitrile molecule disordered over two positions at a percentage occupancy ratio of 53.9 (9):46.1 (9) and one fully occupied and one partially occupied, (25%) benzene molecule, with the latter being located on an inversion centre.

### DFT and MD Simulations

Density Functional Theory optimisation was carried out at the SSBD-D3/LANL2-DZ and SSBD-D3/DEF2-SVP, with the corresponding ECP basis for the metal, using NwChem 6.8.1 software on Bluebear HPC cores.

Parametarisation of the cylinders employed MCPB.py from Amber18 at the wB97XD/6-31G* level of theory in Gaussian09 after initial structure optimisation as described previously. Topologies and coordinates were converted to gromacs using parmed. Production molecular dynamics was run in gromacs2019 with 1 nm electrostatic and coulomb cut-off and 2 fs time step, after minimisation with F max at 500 kJ/mol/nm, 1 ns nvt under constrains and 1 ns npt at 310 K. For DNA parmbsc1 forcefield was used.

During the simulations CB[10] deforms. To evaluate the energy penalty of this deformation we performed DFT optimisation as described above on the CB10 and the deformed CB10 under the same parameters. CB[10] has total DFT energy of −6063.75195199 H whereas the deformed has energy of −6063.73417131H; difference ΔE=46.7 kJ/mol.

### Results and Discussion

The ions exemplified herein are tetracations formed by wrapping three organic ligand strands about two metal centres (see FIG. 2). They are cylindrical in shape, and around 2 nm in length and 1 nm in diameter - greater than the dimensions normally associated with rotaxanation or encapsulation. Cucurbit[10]uril (see FIG. 2) was identified as a macrocycle that might offer a cavity of suitable dimensions (X. R. Yang et al., Chinese Chem. Lett. 2018, 29, 1560-1566).

Cucurbit[n]urils (CB[n]s) are a class of macrocycles that are attracting much attention for both host-guest chemistry and rotaxanation studies (see S. J. Barrow et al., Chem. Rev. 2015, 115, 12320-12326). They are pumpkin-shaped molecules with a rigid hydrophobic cavity flanked by two identical openings (portals) whose rims are each lined with n carbonyl groups. CB[5,6,7] are ideal sizes for traditional small organics and CB[6,7,8] have been used with organic, linear threads and chains to create rotaxanes (see L. Isaacs, Acc. Chem. Res. 2014, 47, 2052-2062). Other applications have included using the host guest chemistry to attach proteins to surfaces or fluorophores, and for drug delivery. CB[10] (FIG. 2) can accommodate multiple or larger molecules, as demonstrated by its use to capture both a Stoddart ‘blue-box’ (bis-bipyridinium) macro-cycle and its methoxybenzene guest to create a Russian-doll style assembly (W. J. Gong, Chem., Eur. J. 2016, 22, 17612-17618).

While lower order cucurbiturils have relatively rigid cavities, CB[10] is more flexible. It is observed to be ellipsoidal in its crystal structure (cavity diameter 1.13-1.24 nm; portal diameter 0.95-1.06 nm) in complex with 4,6-bis(4-(ammoniomethyl)phenylamino)-1,3,5-triazen-2(1H)-one (X. R. Yang, 2018, supra). The previous use of CB[10] for rotaxanation is restricted to a report of a kinetic rotaxane—a pseudo-rotaxane in which the thread is encapsulated at room temperature but can enter and leave at elevated temperatures (see Y. Yu et al., J. Org. Chem. 2017, 82, 5590-6).

To initiate studies, it was first explored whether the supramolecular cylinders would bind to CB[10] to give a tube threaded through a ring. CB[10] itself is an insoluble molecule, but rapidly dissolves on addition of a solution of [M₂L₃]⁴⁺ cylinders (M=Fe, Ru, Ni). Electrospray mass spectrometry shows peaks corresponding to [M₂L₃·CB10]⁴⁺ and proton NMR spectroscopy of [Fe₂L₃]⁴⁺ with CB[10] confirms formation of a 1:1 complex (FIG. 3). DOSY NMR reveals that a single species is formed, of greater hydrodynamic radius than the free cylinder, and involving both cylinder and CB[10]. The cylinder retains its two-fold and three-fold symmetry in the complex. The proton NMR resonances corresponding to the central CH₂ of the cylinder and the adjacent phenylene protons experience upfield shifts, consistent with a shielded location in the CB[10] cavity, while the imine proton and the pyridine H₃ are shifted downfield, consistent with encountering the carbonyls at the rim of the macrocycle. NOEs are observed from these two cylinder protons (Hᵢₘ and H₃) to the CH2 proton on the rim of the CB[10] macrocycle. The NMR is unambiguous confirmation that the cylinder sits symmetrically (on the NMR timescale) in the centre of the macrocycle threading through from one side to the other as a pseudo-rotaxane, with the macrocycle sitting over the central diphenylmethanes of the cylinder as desired. To retain its three-fold symmetry within the ten-fold symmetric CB[10], the cylinder must be rapidly spinning or rocking about the metal-metal axis while within the macrocycle. DFT calculations and MD simulations reproduce this solution structure, with the DFT structure showing axial deviation between the host and guest main symmetry axes at the energy minimum and the MD showing the rotation. Use of a small excess of cylinder allows free and bound cylinder peaks to be observed in NMR spectra (see FIG. 4) indicating that exchange is slow on the NMR timescale, however on mixing [Fe₂L₃]⁴⁺ with [Ni₂L₃·CB10]⁴⁺ and immediately injecting into an electrospray mass spectrometer a mixture of [Fe₂L₃·CB10]⁴⁺ and [Ni₂L₃·CB10]⁴⁺ was observed, the ratio of which remained unchanged over a further hour of sampling. Together these observations indicate that the exchange takes place over a timescale of milliseconds to seconds. The proton NMR spectrum of [Ru₂L₃]⁴⁺ with CB[10] shows analogous upfield and downfield shifts in the resonances and the paramagnetically shifted proton NMR of [Ni₂L₃]⁴⁺ with CB[10] also shows the binding (see FIG. 5).

While the ruthenium(II) and nickel(II) [M₂L₃]⁴⁺ cylinders have excellent stability, the iron(II) cylinders can experience some hydrolytic degradation over long periods (see L. Cardo et al., cellulo Sci. Rep. 2018, 8, 13342 (2018)) though they are stabilised by binding to DNA (see M. J. Hannon et al., Angew. Chem., Intl. Ed., 2001, 40, 879-884). Encapsulation of the cylinder within the CB[10] macrocycle similarly enhances the cylinder stability; an experiment with 0.9 equivalents of CB[10] revealed no degradation of the bound cylinder in aqueous solution over a period of 194 days.

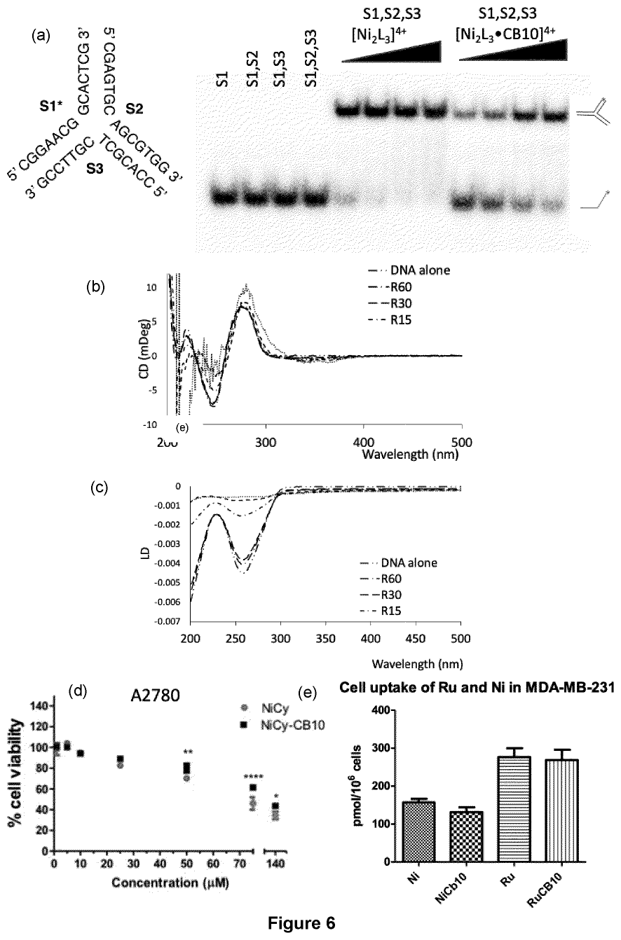

To explore the effect of the encapsulation on the cylinder's DNA-recognition properties the binding of the nickel cylinder·CB[10] complex (as representative of the three complexes) to DNA 3-way junctions (3WJ) was investigated using gel electrophoresis. The assay (FIG. 6a) uses a 3WJ formed from three different complementary DNA strands. The 3WJ structure is (entropically) unstable at room temperature in absence of the cylinder and the DNA exists as three single strands. When the cylinder binds, the 3WJ is stabilised and observed in the gel. Radiolabelling one strand allows a simple on/off gel-shift binding assay that is readily visualised. FIG. 6a shows that the cylinder·CB[10] complex stabilises the 3WJ and the effect of the cylinder·CB[10] complex is the same as the cylinder alone, though higher loadings are required. This indicates that the cylinder can leave the CB[10] cavity and bind to the anionic DNA 3WJ cavity; while binding of an intact cylinder·CB[10] complex to the 3WJ cannot be excluded this would be expected to give a different gel shift.

The binding to calf-thymus DNA (a polymeric genomic DNA), using circular and flow linear dichroism spectroscopies (FIG. 6b,c) was also studied. The effects are again the same as the free cylinder binding: The circular dichroism confirms binding of cylinder (an induced CD signal is observed in the cylinder spectroscopy between 300-400 nm) and retention of a B-DNA structure (typical signature between 200-300 nm). The CD spectra are the same as those observed with cylinder alone, although at high loading some scattering is observed consistent with release of CB[10] which (uncomplexed) will be insoluble. The flow linear dichroism experiments probe the orientation of ct-DNA in a couette flow cell, with the magnitude of the peak at 260 nm reflecting the orientation (see see M. J. Hannon et al., 2001, supra). Cylinders are known to coil ct-DNA (leading to a loss of orientation); the spectra on addition of the cylinder·CB[10] complex confirm the same DNA-coiling effects and are analogous to those observed with free cylinder. We conclude that while the CB[10] does bind the cylinder in solution to form the pseudo-rotaxane, the cylinder preferentially binds to anionic DNA. For the 3WJ, which is not pre-formed in solution, the extra energy of forming the structure means that there is competition between binding CB[10] or 3WJ. When presented with a polymeric DNA anion the cylinder leaves the CB[10] cavity and binds its preferred partner.

Consistent with the in vitro DNA binding, when we treat cells (A2780 ovarian and MDA-MB-231 breast cancer lines) with the cylinder·CB[10] complex, we observe very similar inhibitions of cell proliferation (as assessed by an MTT assay) to those with the corresponding free cylinder (FIG. 6). There is a very slight enhancement in activity for the ruthenium cylinder but a slight drop in activity for the iron and nickel cylinders. Uptake studies (assessed by ICP-MS) indicate that the encapsulation inside CB[10] has little effect on the extent of overall uptake (FIG. 6). The DNA-binding studies indicate that whether the cylinders have entered alone or in complex with CB[10], their effects when they reach the target DNA are expected to be the same.

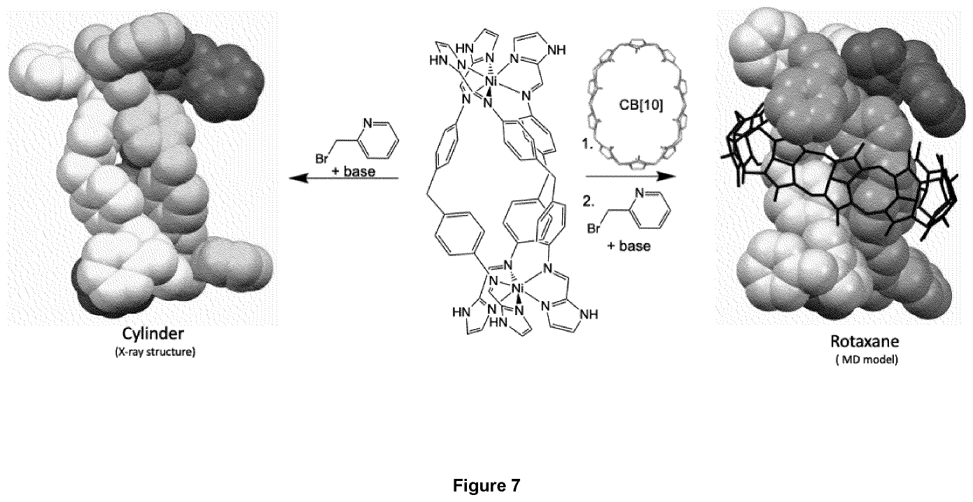

Having established that pseudo-rotaxanation with CB[10] was readily achieved and that it placed the macrocycle in the correct position over the central section of the cylinder but that it had little effect on the biological properties due to competitive de-threading, we next explored if we could lock the macrocycle onto the cylinder as a proper rotaxane. Traditional rotaxanes based on linear (1 D or 2D) threads, are formed by attaching two bulky stoppers, one at each end of the thread. By using a 3-dimensional tube (cylinder) as the axle we open up an alternative possibility for locking the ring onto the structure, by attaching multiple (smaller) groups that protrude out of the cylindrical surface at the ends of the axle (like branches/roots from a tree trunk). To explore this we modified the design of the cylinder, replacing the pyridines with 2-imidazole units. The 2-imidazole offers a position on the cylindrical surface to which we can add further functionalisation, through alkylation of the non-coordinated nitrogen in the imidazole ring, and we selected 2-picolyl as the alkylating group. Thus rather than introducing a single bulky stopper, we introduce three branch points onto the cylinder surface at each end. This new design approach is shown schematically in FIG. 1 and structurally in FIG. 7. While the structure of a traditional rotaxane is often likened to a ring around a dumb-bell, this new type of rotaxane design is more akin to a ring wrapped around the trunk of a tree with the branches and roots acting as stoppers.

We first prepared two non-rotaxanated cylinders, [Ni₂L′₃]⁴⁺ and [Ni₂L″₃]⁴⁺, analogous to the parent cylinders but bearing the 2-imidazole and 2-imidazole-N-picolyl groups in place of the pyridines. They are readily prepared from the condensation of the 2-imidazole and 2-imidazole-N-picolyl carboxaldehdyes with bis-1,4-diaminophenylmethane and subsequent coordination to nickel(II). While the corresponding iron(II) complexes were also prepared, [Fe₂L′₃]⁴⁺ had a lower solution stability than the corresponding nickel(II) complex and so we focused our studies on the nickel complexes. The X-ray crystal structures of the [M₂L″₃]⁴⁺ cations (M=Ni, Fe) were obtained (as the hexafluorophosphate salts) and confirmed the expected structure (FIG. 7), with the retention of the triple-helical cylindrical structure, the retention of the central core of the cylinder structure which is important for its binding to both DNA 3-way junctions and CB[10], and now the introduction of the picolyl units sticking out of the helix and in such a position that they should not interfere with the CB[10] binding site but would potentially sterically prevent it from threading/dethreading.

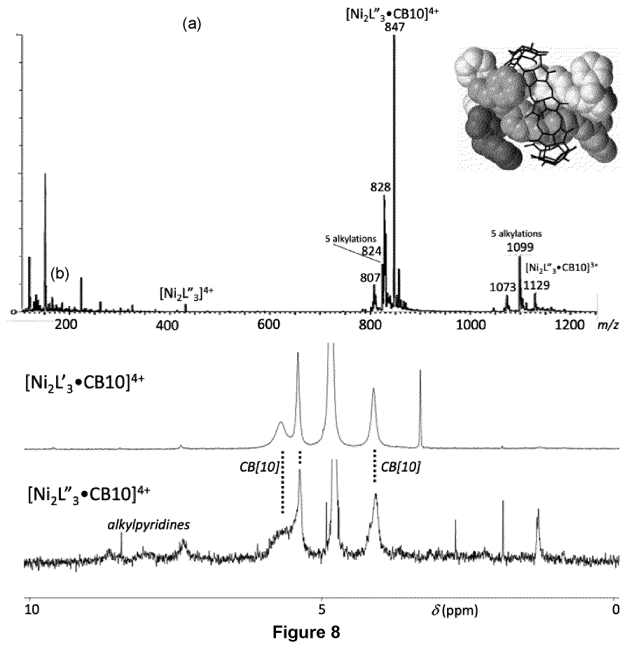

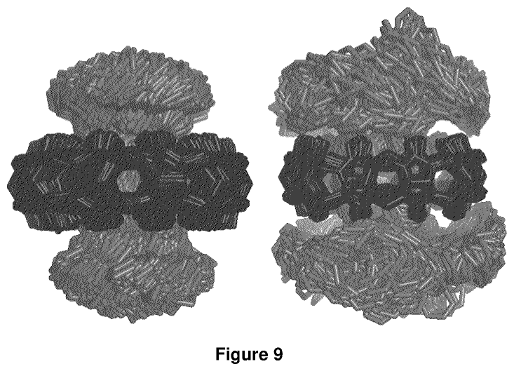

We then explored whether we could post-synthetically transform [Ni₂L′₃]⁴⁺ into the alkylated [Ni₂L″₃]⁴⁺ by reaction with alkylating agent 2-(bromomethyl)pyridine and Hunig's base in acetonitrile; heating at reflux overnight afforded the desired hexa-alkylated product, [Ni₂L″₃]⁴⁺, as confirmed by mass spectrometry and (paramagnetic) proton NMR spectroscopy. Small amounts of the penta-functionalised cylinder were also observed by mass spectrometry. To access the rotaxane we then repeated the same reaction in the presence of CB[10]. While acetonitrile did not prove to be a suitable solvent, reaction in aqueous methanol at reflux overnight, afforded the rotaxane as a cream precipitate in ˜40% isolated yield. The mass spectrum (FIG. 8) shows a dominant peak corresponding to [Ni₂L″₃·CB10]⁴⁺ (correct mass and isotope distribution). Smaller peaks corresponding to penta-alkylated rotaxanes are also observed but only a tiny trace peak corresponding to un-rotaxanated [Ni₂L″₃]⁴⁺ indicating that the rotaxane (as opposed to a pseudo-rotaxane) is formed in which the ring is now constrained by the picolyl groups to remain on the cylinder. The (paramagnetically shifted) proton NMR spectrum shows the presence of peaks corresponding to the cylinder and the CB[10] (FIG. 8). The cylinder peaks in the rotaxane are further broadened than those observed in the free [Ni₂L″₃]⁴⁺ cylinder; consistent with this, MD simulations (FIGS. 7 and 8) indicate that the picolyl groups restrict the spinning motion of the cylinder about the metal-metal axis while rotaxanated inside the CB[10]. The rotaxanated complex [Ni₂L″₃·CB10]⁴⁺ was mixed with an equimolar amount of [Fe₂L₃]⁴⁺ and heated under reflux for 24 hours in 10% methanol: water. The rotaxane was unchanged and no Fe₂L₃ pseudo-rotaxanes were observed by mass spectrometry; this contrasts with the analogous mixing experiments with pseudo-rotaxanes and is strong evidence of proper rotaxane formation.

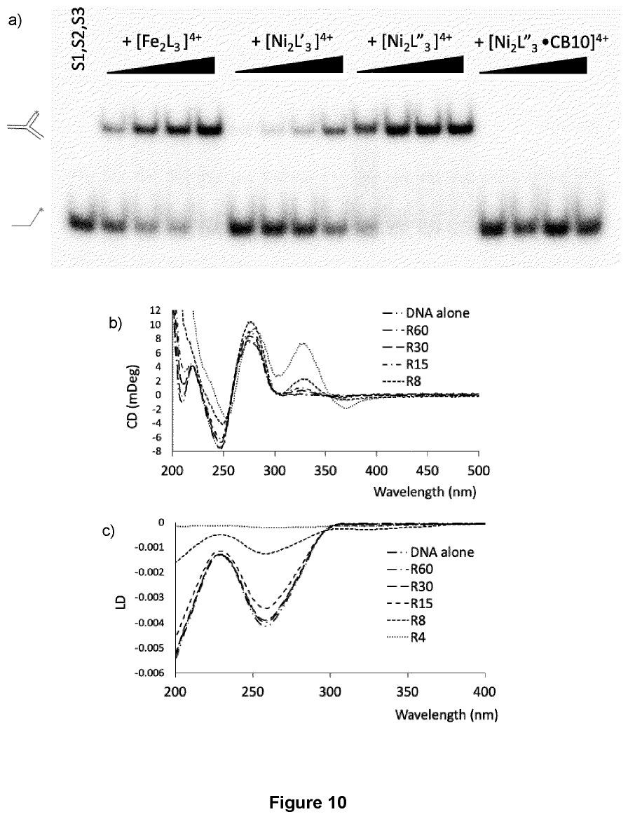

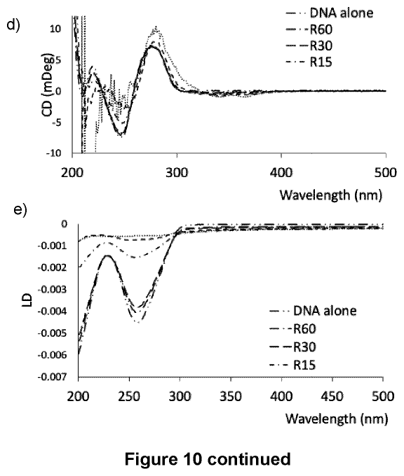

The non-rotaxanated [Ni₂L′₃]⁴⁺ and [Ni₂L″₃]⁴⁺ cylinders bind DNA analogously to the original pyridyl cylinders (FIG. 10). PAGE gel electrophoresis experiments confirms that both are able to bind and stabilise DNA 3-way junctions, just as the original pyridyl cylinders do; circular dichroism spectra confirm that both cylinders bind calf thymus DNA (inducing a CD signal in the cylinder spectroscopy) which retains a B-DNA structure, and the linear dichroism demonstrates that both cylinders give rise to DNA-coiling as observed with pyridyl-based cylinders. Cell proliferation (MTT) assays confirmed that the functionalised (but non-rotaxanated) cylinder [Ni₂L″₃]⁴⁺ exerts a cytotoxic effect in cells (IC₅₀, 72 h, MDA-MB-231 55±10 μM; SKOV3 52±5 μM). Thus, as anticipated, making changes to the ends of these cylinders does not, of itself, prevent the key DNA-binding and biological activities.

The contrast with the rotaxane [Ni₂L″₃·CB10]⁴⁺ is striking (FIG. 10). The rotaxane can no longer stabilise the DNA 3WJ in the gel electrophoresis experiment. It also no longer coils DNA (rather a small increase in DNA orientation is observed). Thus wrapping the CB[10] macrocycle around the central phenylene units has switched off the DNA junction binding and DNA coiling. The greater DNA orientation observed in a couette cell in the linear dichroism experiment indicates some form of DNA binding occurs, while the circular dichroism confirms that the DNA remains in its B form. Molecular dynamics simulations suggest that the rotaxane may associate through a partial entry into the grooves, and that this leads to some small elongation of the DNA. Association with the bases at the ends of the DNA strand is also seen in the simulations together with some capacity to bridge across two pieces of DNA that could also lead to enhanced orientation in a couette cell.

Just as the rotaxanation switches off the key DNA binding features of the cylinders, it also switches off the biological action with the rotaxane showing no cytotoxic effect in cell proliferation (MTT) assays (IC₅₀, 72 h>>140 μM) in MDA-MB-231 (breast) cell lines.

The above results support the creation of systems in which a triggered rotaxanation or de-rotaxanation (de-threading) inside a cell might be used to switch a metallo-drug action on or off.

### Temporal Control of Rotaxane Release

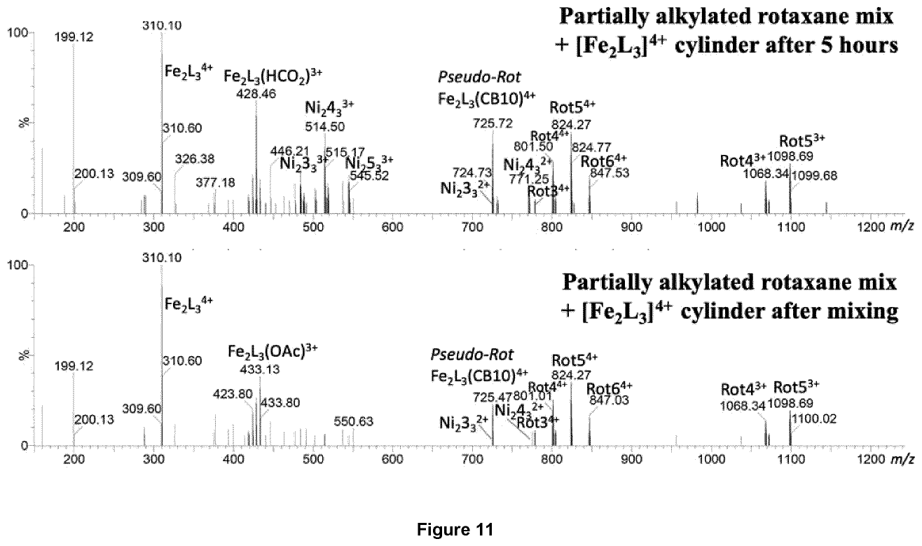

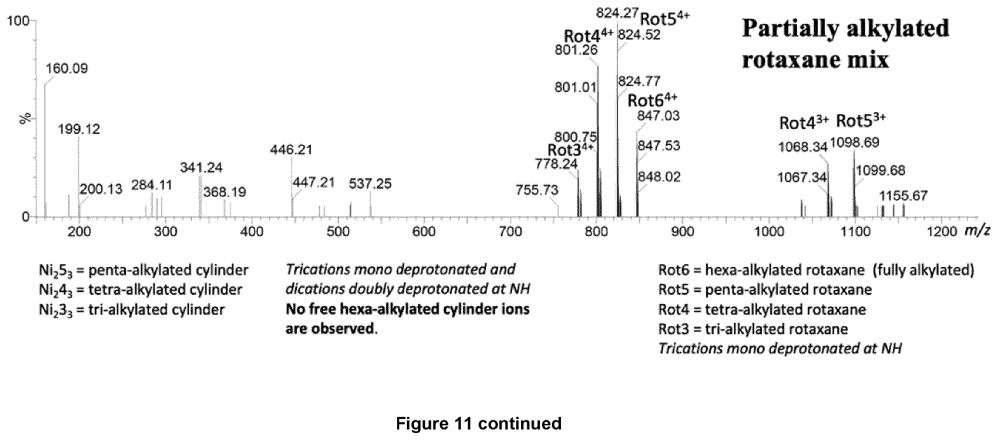

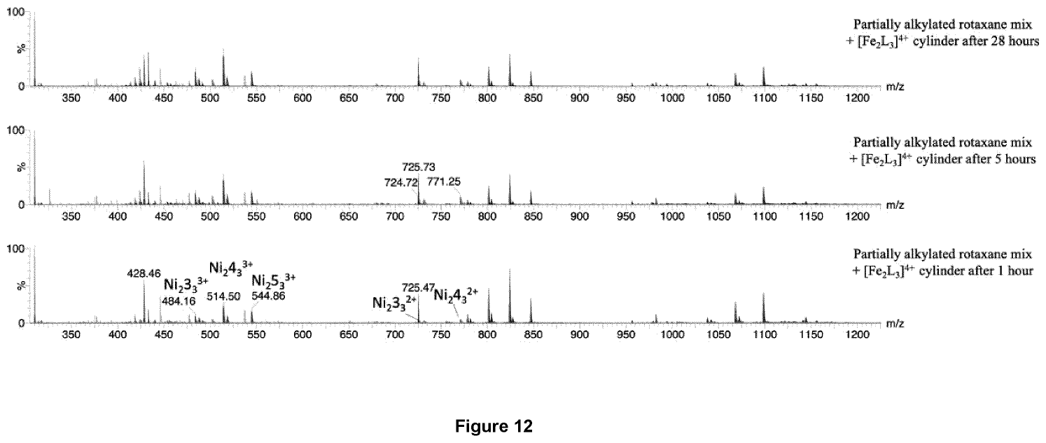

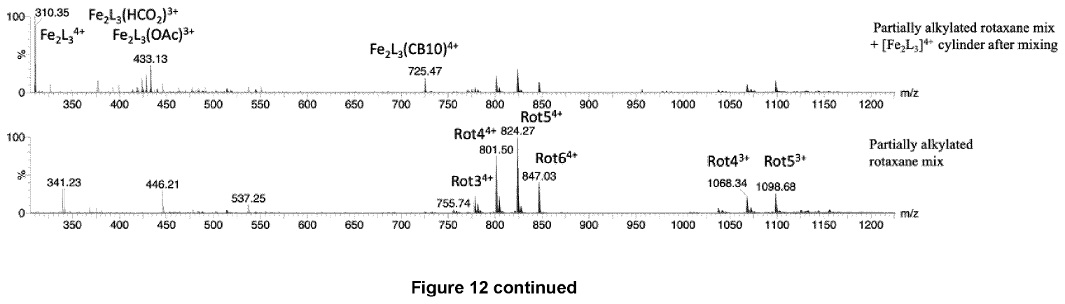

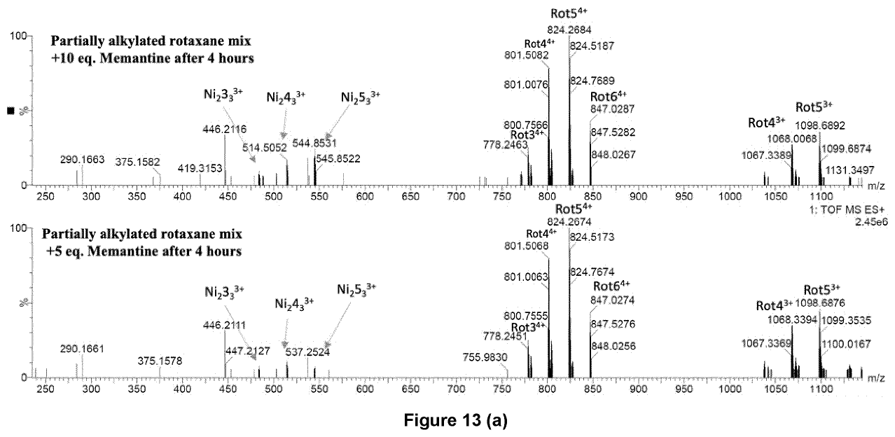

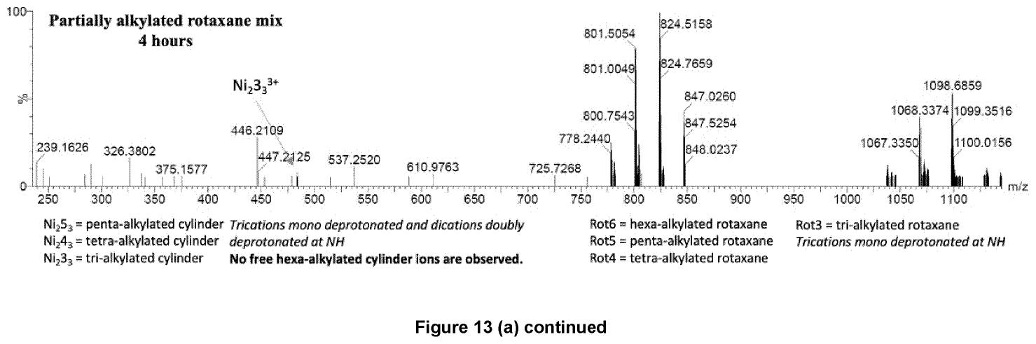

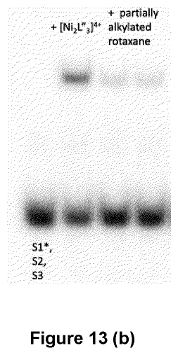

To explore thread release we prepared a rotaxane mixture in which we only partially alkylated the rotaxane thereby varying the number of picolyl branches/stoppers on the species, and creating a mixture of rotaxanes containing 3-, 4-, and 5- stoppers on the thread as well as the fully-stoppered 6-alkylated rotaxane (FIG. 11). We then undertook a competition experiment with the [Fe₂L₃]⁴⁺ cylinder (FIGS. 11 and 12). The results are consistent with a kinetic de-rotaxanation for cylinders that are not fully stoppered: the competitor initially displaces tri- and tetra-alkylated cylinders from the CB[10] ring and after further time also penta-alkylated cylinders. No peaks corresponding to displacement of hexa-alkylated (fully alkylated) cylinders are observed even over long time periods (28 h). An analogous experiment with 5 and 10 equivalents of competitor guest memantine (FIG. 13a) showed similar effects.

The tri-alkylated cylinders, at one end bear just one or no stoppers, and the tetra-alkylated cylinders have an end with either one or two stoppers. The penta-alkylated cylinder has one end with two stoppers, while in the hexa-alkylated cylinder both ends contain three stoppers. The results imply that cylinders with a mono-alkylated end de-thread more readily than those whose ends are di-alkylated, consistent with the greater steric barrier to de-threading that increased alkylation should present.

This poorly alkylated rotaxane mixture also gave rise to a weak 3WJ band in gel electrophoresis (FIG. 13b), consistent with a small proportion of poorly alkylated cylinders dethreading from the CB[10] and binding instead to the 3WJ. These results provide a demonstration of how the extent and type of branch-point alkylation can be used to control the kinetics of de-rotaxanation and in turn kinetically and temporally modulate release of the 3WJ-binding drug.

### Alternative Synthetic Route to the Rotaxanes

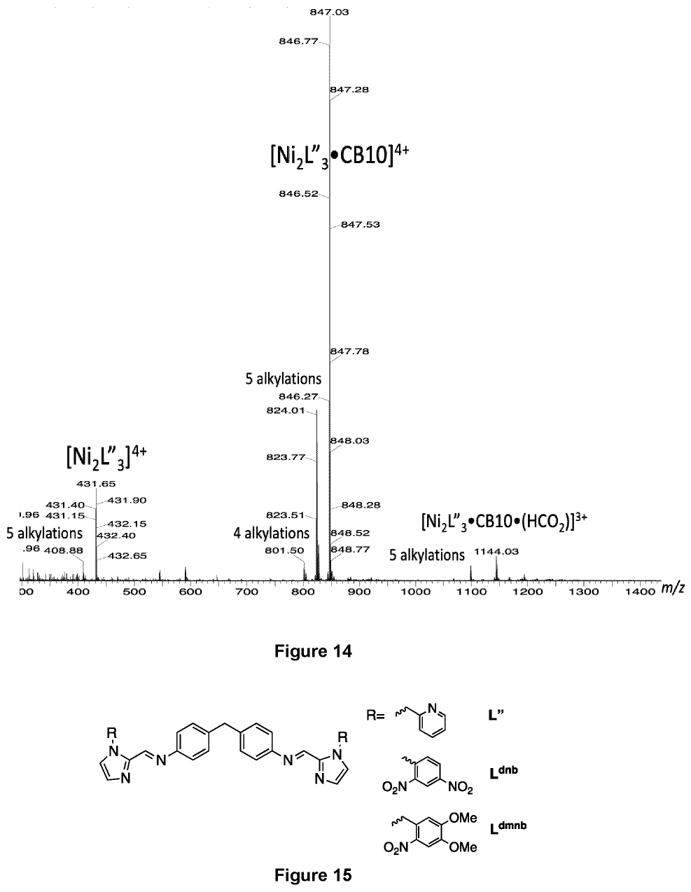

As an alternative synthetic route, the same [Ni₂(L″)₃·CB10]⁴⁺ rotaxane can be prepared in almost quantitative yield by heating the pre-alkylated [Ni₂L″₃]⁴⁺ in aqueous solution with CB[10] at 100° C. for 16 hours. The formation of the rotaxane species is confirmed by mass spectrometry (FIG. 14). The analogous iron(II) rotaxane [Fe₂(L″)₃·CB10]⁴⁺ could be prepared by the same method heating at 50° C. for 1 h.

Similar [Ni₂(L^{dmnb})₃·CB10]⁴⁺ rotaxanes can also be prepared based on the ligand L^{dmnb} (FIG. 15) in which the picolyl unit of L″ is replaced with a dimethoxynitrobenzyl unit. Both synthetic routes can be used for the compound preparation.

### De-Capping and De-Rotaxanation in Response to Thiols

The rotaxane [Ni₂(L^{dnb})₃·CB10]⁴⁺ was prepared in an analogous way to the rotaxane [Ni₂(L″)₃·CB10]⁴⁺ rotaxane using 1-fluoro-2,4-dinitrobenzene to introduce 2,4-dinitrobenzene caps at the imidazole of the Ni₂(L′)₃·CB10]⁴⁺ pseudo-rotaxane.

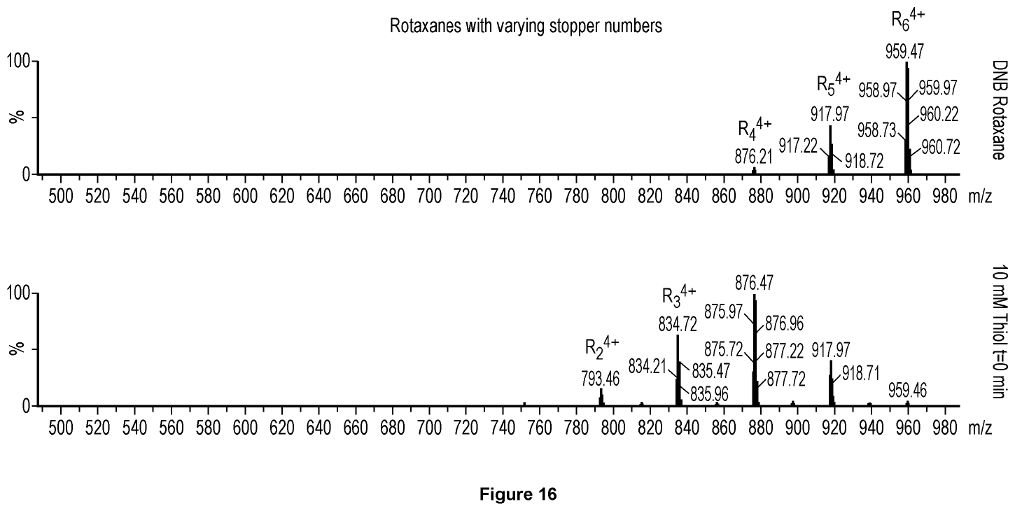

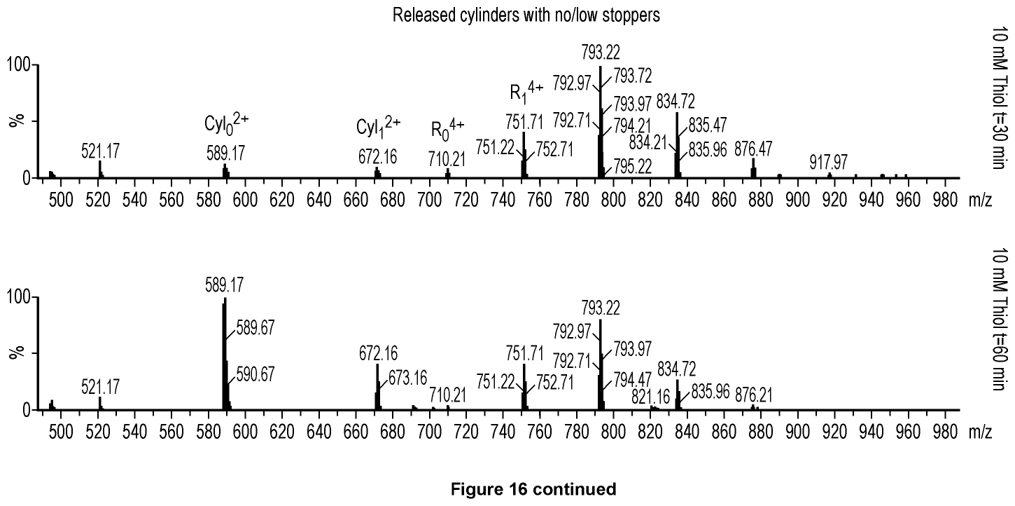

The capping groups can be removed by thiols. To demonstrate this, the rotaxane [Ni₂(L^{dnb})₃·CB10]⁴⁺ was treated with 10 mM mercaptoethanol in water and the solution species monitored by electrospray mass spectrometry (FIG. 16). Over 60 minutes sequential losses of caps from the rotaxane were observed together with the presence of free cylinder peaks as the cylinder is then released from the CB[10] ring. Similar decapping and release effects were achieved with glutathione and NaSH. The rotaxane is stable in the absence of the thiols.

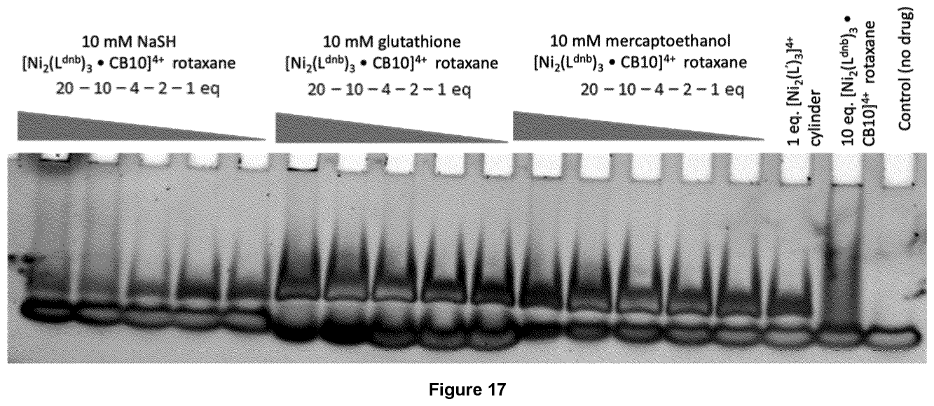

To confirm that the released cylinder is now able to bind DNA-junctions, we investigated the binding of the [Ni₂(L^{dnb})₃·CB10]⁴⁺ rotaxane in absence and in presence of mercaptoethanol to DNA 3-way junctions (3WJ) using gel electrophoresis. The assay (FIG. 17) uses a 3WJ formed from three different complementary DNA strands. The 3WJ structure is (entropically) unstable at room temperature in absence of free cylinder, and the DNA exists as three single strands. When the cylinder binds, the 3WJ is stabilized and observed in the gel. Radiolabelling one strand allows a simple on/off gel-shift binding assay that is readily visualized. FIG. 16 shows that the intact capped rotaxane [Ni₂(L^{dnb})₃·CB10]⁴⁺ does not stabilize the 3WJ, but that when treated with decapping agent mercaptoethanol, the caps are released, the cylinder can leave the CB[10] cavity and bind to the DNA 3WJ as anticipated.

1. A rotaxane comprising a macrocycle and an ion of formula [M_{z}L_{z1}]^{zn+}, wherein M is a metal ion or combination of ions of oxidation state n⁺, z is 2 to 4, z1 is 2 to 6 and L is a ligand of formula (I): 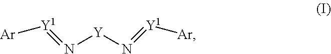
wherein Y¹ is independently CR¹ or N;
R¹ is independently selected from H, C₁-C₄alkyl, amino, phenyl or C₃-C₅heteroaryl, wherein the phenyl or C₃-C₅heteroaryl is optionally substituted with any one or a combination selected from the group consisting of C₁-C₄alkoxy, C₁-C₄alkyleneC₁-C₄alkanoate, C₁-C₄alkanoate, hydroxy, C₁-C₄alkylol, carboxy, C₁-C₄alkyl, halo, cyano, nitrite, C₁-C₄haloalkyl, C₁-C₄alkylthio, C₁-C₄alkyleneC₁-C₄alkylthio, C₁-C₄alkylsulfonate, diC₁-C₄alkylamino and C₁-C₄alkynyl;
each Ar is positioned at either end of the ion of formula [M_{z}L_{z1}]^{zn+} and is independently a C₃-C₉heteroaryl comprising at least one nitrogen atom, is optionally substituted with one or more blocking groups capable of trapping the macrocycle, with the proviso that each end of the ion comprises at least one blocking group, and is optionally substituted with one or more substituents selected from the group consisting of hydroxy, C₁-C₄alkylol, carboxy, C₁-C₄alkanoate, C₁-C₄alkyl, C₁-C₄alkoxy, halo, cyano, nitrite, C₁-C₄haloalkyl, C₁-C₄alkylthio, C₁-C₄alkyleneC₁-C₄alkylthio, C₁-C₄alkylsulfonate, diC₁-C₄alkylamino and C₁-C₄alkynyl;
N—Y—N is N—N or is any one selected from the group consisting of (Ia) to (Ic): 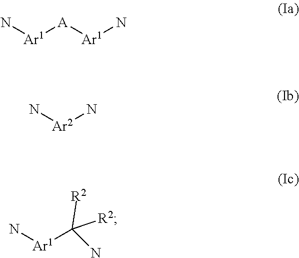
wherein A is NH, S, SO₂, O, (CH₂)₁₋₄, CHR², CR²₂ or NR²;
Ar¹ is phenyl optionally substituted one or more times with R³;
Ar² is phenyl or biphenyl optionally substituted one or more times with R³; and
R² and R³ are independently any one or a combination selected from the group consisting of H, hydroxy, C₁-C₄alkylol, carboxy, C₁-C₄alkanoate, C₁-C₄alkyl, halo, cyano, nitrite, C₁-C₄haloalkyl, C₁-C₄alkylthio, C₁-C₄alkyleneC₁-C₄alkylthio, C₁-C₄alkylsulfonate, diC₁-C₄alkylamino and C₁-C₄alkynyl.

2. The rotaxane of claim 1, wherein each Ar is substituted with at least one blocking group capable of trapping the macrocycle.

3. The rotaxane of claim 1 or claim 2, wherein N—Y—N is represented by formula (Ia).

4. The rotaxane of any one of claims 1 to 3, wherein A is:
(i) (CH₂)₁₋₄, O, NH, CHR², CR²₂ or NR²; or
(ii) CH₂.

5. The rotaxane of any one previous claim wherein R³ is H or C₁-C₄alkyl and/or R¹ is H.

6. The rotaxane of any one previous claim wherein each Ar is optionally substituted with any one or a combination selected from the group consisting of C₁-C₄alkanoate and C₁-C₄alkyl.

7. The rotaxane of any one previous claim wherein each Ar is independently an optionally substituted 2-imidazolyl, 5-imidazolyl, 2-pyridyl, 2-quinolinyl, 2-pyrazinyl, 1-isoquinolinyl and 3-isoquinolinyl.

8. The rotaxane of any one previous claim wherein the blocking groups are capable of reversibly trapping the macrocycle and/or are removable by catalytic hydrogenation, photo-irradiation, enzymatic cleavage, reduction or hydrolysis.

9. The rotaxane of any one previous claim wherein the blocking groups are any one or a combination selected from the group consisting of C₃-C₅heteroarylC₁-C₄alkylene, phenylC₁-C₄alkylene, propargyl, 1-(propyne-1,3-diyl)C₁-C₄alkyl, 2-propyn-1-yl formate, 3-C₁-C₄alkyl-2-propyn-1-yl formate, formate, C₁-C₄alkyl formate, benzyl formate, propargyloxybenzyl formate, 2-nitrophenyl formate, 2-nitrobenzyl, 2-nitrobenzyl carbonate, phenacylcarboxylate, coumarin-4-ethanoate, dinitrophenyl, p-galactoside, glucuronide, glutamate, 2-aminoC₁-C₄alkyl-glutamate, wherein the 2-nitrophenyl formate, 2-nitrobenzyl, 2-nitrobenzyl carbonate, phenacylcarboxylate and coumarin-4-ethanoate are optionally substituted with one or more substitutents selected from the group consisting of C₁-C₄alkoxy, diC₁-C₄alkoxyphenyl, hydroxy, halo, amino and carboxy.

10. The rotaxane of claim 9, wherein the optionally substituted 2-nitrophenyl formate, is 4,5-dimethoxy-2-nitrophenyl formate, the optionally substituted 2-nitrobenzyl is 4,5-dimethoxy-2-nitrobenzyl, the optionally substituted 2-nitrobenzyl carbonate is 4,5-dimethoxy-2-nitrobenzyl carbonate and the optionally substituted phenacylcarboxylate is 2-(3,5-dimethoxyphenyl)-2-carboxy-1-phenylethanone.

11. The rotaxane of claim 1 wherein L is of formula (IIa): 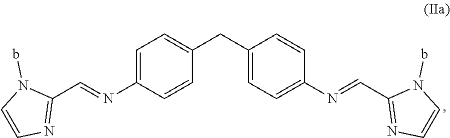
wherein each b is optionally present and is a blocking group, as defined in any one of claims 1 and 10 to 14, with the proviso that each end of the ion comprises at least one blocking group.

12. The rotaxane of claim 1 wherein L is of formula (IIIa):

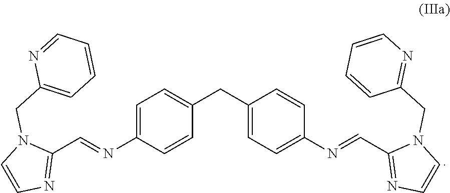 [SMILES: C(=N\c1ccc(Cc2ccc(/N=C/c3nccn3Cc3ccccn3)cc2)cc1)/c1nccn1Cc1ccccn1]

13. The rotaxane of any one previous claim wherein M is a metal ion of any one metal or a combination of metals selected from the group consisting of:
(i) Fe, Ni, Ru, Co, Cu, Ag, Cd, Zn, Rh, Mn, Ir, Os, Pd, and Pt; or
(ii) Fe, Ni and Ru.

14. The rotaxane of any one preceding claim wherein n+ is 2+.

15. The rotaxane of any one preceding claim wherein z is 2 and z1 is 2 or 3.

16. The rotaxane of any one preceding claim wherein the macrocycle is:
(i) any one selected from the group consisting of a cucurbituril, crown ether, cyclodextrin, calixarene, pillararene, and metallo-organic macrocycle, or the macrocycle comprises a porphyrin, corrin, chlorin or aryl groups linked by C₁-C₄alkylenes, C₁-C₄ethers, C₂-C₄alkenylenes and C₂-C₄alkynylenes; or
(ii) cucurbit[10]uril.

17. A method of synthesising a rotaxane, the method comprising:
(i) contacting a macrocycle and an ion of formula [M_{z}L_{z1}]^{zn+} to produce a pseudo-rotaxane, wherein the ion of formula [M_{z}L_{z1}]^{zn+} is as defined in claim 1, with the proviso that each Ar is not substituted with one or more blocking groups capable of trapping the macrocycle; and
(ii) reacting the pseudo-rotaxane with a compound of formula b-X to form the rotaxane, wherein b is a blocking group capable of trapping the macrocycle and X is a leaving group, with the proviso that each end of the ion of the rotaxane comprises at least one blocking group.

18. The method of claim 17 wherein the macrocycle is as defined in claim 16, [M_{z}L_{z1}]^{zn+} is as defined in any one of claims 2 to 7, and 13 to 15 and/or b is as defined in any one of claims 8 to 10.

19. The method of claim 17 or claim 18 wherein X is:
(i) any one selected from the group consisting of halo, C₁-C₄alkylsulfonate, C₁-C₄haloalkylsulfonate and phenylsulfonate optionally substituted one or more times with any one or a combination from the group consisting of C₁-C₄alkyl and C₁-C₄haloalkyl; or
(ii) bromo or iodo

20. A method of synthesising a rotaxane, the method comprising contacting a macrocycle and an ion of formula [M_{z}L_{z1}]^{zn+} at temperatures of about 50 to about 100° C., wherein the ion of formula [M_{z}L_{z1}]^{zn+} is as defined in claim 1.

21. The method of claim 20, wherein the macrocycle is as defined in claim 16 and/or [M_{z}L_{z1}]^{zn+} is as defined in any one of claims 2 to 15.

22. A method of removing blocking group(s) from the rotaxane of any one of claims 1 to 16, the method comprising reacting the rotaxane with hydrogen, reacting the rotaxane with a thiol, photo-irradiating the rotaxane, contacting the rotaxane with an enzyme, reacting the rotaxane with a reducing agent or reacting the rotaxane with water.

23. The method of claim 22 wherein the reacting the rotaxane with hydrogen is in the presence of a hydrogenation catalyst, such as palladium, and/or the enzyme is any one selected from the group consisting of a nitrophenylreductase, galactosidase, carboxypeptidase, β-lactamase and β-glucuronidase.

24. An ion of formula [M_{z}L_{z1}]^{zn+}, as defined in claim 1, wherein the blocking groups are capable of reversibly trapping a macrocycle.

25. The ion of claim 24, wherein the ion is as defined in any one of claims 2 to 7 and 8 to 15.

26. A pharmaceutical formulation comprising the rotaxane of any one of claims 1 to 16 and one or more pharmaceutically acceptable excipients.

27. A rotaxane of any one of claims 1 to 16 or a pharmaceutical formulation of claim 26 for use as a medicament.

28. A rotaxane of any one of claims 1 to 16 or a pharmaceutical formulation of claim 26 for use in the treatment of any one or a combination selected from the group consisting of cancer, a viral disease or a bacterial disease.

29. A method of treatment comprising administering a therapeutically effective amount of a rotaxane of any one of claims 1 to 16 or a pharmaceutical formulation of claim 26 to a subject.

30. A method of treating cancer, a viral disease and/or a bacterial disease comprising administering a therapeutically effective amount of a rotaxane of any one of claims 1 to 16 or a pharmaceutical formulation of claim 26 to a subject.
